(19) United States
(12) Patent Application Publication
(10) Pub. No.: US 2026/0308317 A1
(43) Pub. Date: Oct. 8, 2026

(54) SYNTHETIC WAX MATERIALS WITH DEGRADABLE LINKERS

(71) Applicant: BOARD OF TRUSTEES OF MICHIGAN STATE UNIVERSITY, East Lansing, MI (US)

(72) Inventors: Muhammad Rabnawaz, Okemos, MI (US); Hazem Mohamed Mousa Mohamed Elkholy, East Lansing, MI (US); Ajmir Khan, East Lansing, MI (US); Syeda Shamila Hamdani, Lansing, MI (US); Sarla Yadav, East Lansing, MI (US)

(21) Appl. No.: 19/473,535

(22) PCT Filed: Apr. 12, 2024
(86) PCT No.: PCT/US2024/024191

§ 371 Date: Oct. 8, 2025

Related U.S. Application Data
(60) Provisional application No. 63/458,812, filed on Apr. 12, 2023.

(51) Int. Cl. C08G 63/91; C08J 11/16; C09D 167/04; D21H 19/10

(52) U.S. Cl. CPC C08G 63/91; C08J 11/16; C09D 167/04; D21H 19/10

(57) ABSTRACT

The disclosure relates to synthetic wax compositions including at least one long-chain hydrocarbon residue having 12 to 40 carbon atoms, and at least one oligomeric residue containing 4 to 20 or 4 to 50 glycolic acid units and/or lactic acid units. The synthetic wax can include other functional groups such as pendant carboxylic groups to facilitate emulsification and/or metal complexation of the wax, and/or unsaturated carbon-carbon double bonds to facilitate curing or crosslinking of the wax. The synthetic wax can be used as a coating on a variety of substrates, for example paper substrates, to impart water and/or oil resistance to the substrate. The presence of the oligomeric glycolic/lactic residue in the wax permits chemical degradation under very mild conditions, which in turn facilitates removal, repulping, and/or recycling of the synthetic wax.

### CROSS REFERENCE TO RELATED APPLICATION

Priority is claimed to U.S. Provisional Application No. 63/458,812, filed Apr. 12, 2023, which is incorporated herein by reference in its entirety.

### STATEMENT OF GOVERNMENT INTEREST

This invention was made with government support under RC114052 awarded by the National Science Foundation. The government has certain rights in the invention.

### BACKGROUND OF THE DISCLOSURE

### Field of the Disclosure

The disclosure relates to synthetic wax compositions including at least one long-chain hydrocarbon residue having 12 to 40 carbon atoms, and at least one oligomeric residue containing 4 to 20 or 4 to 50 glycolic acid units and/or lactic acid units. The presence of the oligomeric glycolic/lactic residue in the wax permits chemical degradation under very mild conditions, which in turn facilitates removal, repulping, and/or recycling of the synthetic wax.

### Background

Approximately 40% of all plastics produced today are used in the packaging sector. The biggest sustainable packaging hurdle is access to new materials that are universally compostable, repulpable/recyclable available at commodity prices, and have performance matching or exceeding those of the existing polymers.

Waxes can be used as a paper coating to impart some degree of water and oil resistance to the paper. Petrochemical waxes are difficult to separate from coated paper and thus need alternatives to ensure wax coated paper is recyclable. Natural wax such as beeswax and carnauba wax are good alternatives, but they are expensive and limited in amount.

The environmental concerns related to plastics have shifted momentum toward paper-based packaging. A major concern with plastics is their potential to form microplastics. As an alternative to plastic packaging, paper packaging is in high demand across food and pharmaceutical packaging. This is due to paper's biodegradability, affordability, lightweight, and biobased nature. However, paper in its uncoated form does not fulfill packaging needs. For example, paper is usually coated with various materials to improve its resistance against water, gas, moisture, and oil, as well as to provide thermal sealing properties. However, the challenges are that the coating materials used today are synthetic, nonbiodegradable, and non-repulpable, thus posing a risk to the environment. For example, polyethylene (PE) is widely used for paper coating to fulfill packaging needs. However, the difficulty in separating the paper from these coated materials causes the paper to lose both its recyclable and biodegradable properties. Consequently, coated or laminated paper often ends up in landfills, where it also leaks into rivers. In the case of plastic leakage into the environment, it eventually turns into microplastics and becomes a high risk for both human and ecological health due to mechanical abrasion brought on by water waves and ultraviolet (UV) exposure.

Biodegradable polymers offer potential alternatives to plastics or plastic-coated paper as they do not create any persistent microplastics. Starch, cellulose, and some polyesters are examples of biodegradable/compostable polymers due to the presence of hydrolyzable ester bonds in their structures. Some examples of biodegradable or compostable polymers include polyhydroxy hexanoate (PHH), polyhydroxy butyrate (PHB), polyhydroxy valerate (PHV), polylactic acid (PLA), and polycaprolactone (PCL). PLA is an aliphatic polyester that is industrially synthesized via the ring-opening polymerization of lactide especially for obtaining high-molecular-weight PLA. PLA is safe for all food packaging applications and is categorized by the US Food and Drug Administration (FDA) as generally regarded as safe. However, high-molecular-weight PLA is non-biodegradable in soil as it takes 500 and 1000 years to break down, as PLA only degrades in industry compost conditions.

As part of our continuous efforts to develop sustainable packaging materials, our lab is also actively working on creating coating materials that are recyclable, biodegradable, and effective at providing surfaces that are resistant to oil and water. We have demonstrated a strategy in which chitosan-graft-PDMS (chitosan-g-PDMS) is used to fabricate a coating that is resistant to grease and water.4 However, the limited availability and high cost of chitosan limit its applicability in packaging. We have also reported a plant oil-based coating approach that is eco-friendly, nontoxic, and biodegradable. Since soybean oil has a chemical structure that allows it to change chemically, and thus can be used for paper coating applications.

Wax-coated paper can offer an excellent sustainable alternative to plastics and plastic-coated paper. For example, natural waxes are biodegradable, and thus, they do not generate microplastics. However, waxes are suitable only for low-temperature applications because of their low melting temperature Tm. Also, natural waxes are expensive and insufficiently available for large-scale use in the packaging industry. Also, waxes do not offer good thermal sealing performance because of shorter hydrocarbon chains lacking the necessary polymer chain entanglement.

### SUMMARY

In one aspect, the disclosure relates to a synthetic wax according to the following Formula I: A-a-B-b-C (I). In Formula I, A is a hydrocarbon ester group having 12 to 40 carbon atoms; B is an oligomeric residue of (i) n glycolic acid units, (ii) n lactic acid units, or (iii) n glycolic acid units and lactic acid units in total, where n is 4 to 20 (or 4 to 50, or 6 to 14); C is either OH or a hydrocarbon ester group having 2 to 40 carbon atoms, with the proviso that when C is OH, then A contains at least one of a carboxylic group and an unsaturated carbon-carbon double bond; a is either absent, or present as a linking group between A and B; and b is either absent, or present as a linking group between B and C.

In Formula I, A can include linear, branched, or cyclic, saturated or unsaturated, substituted or unsubstituted hydrocarbon groups having at least one ester functional group. For example, A can include linear, branched, or cyclic, substituted or unsubstituted alkyl or alkenyl (e.g., having one or more C═C unsaturated bonds) groups with a terminal ester functional group at the point of bonding to a or B. The A group can be substituted with or otherwise include a carboxylic group (e.g., in acid or salt form) or an unsaturated (polymerizable) C═C unsaturated bond. The relatively long hydrocarbon character of A imparts at least some of the waxy character to the synthetic wax. The A group can include at least 8, 12, 14, 16, 18, 20, or 24 and/or up to 16, 20, 24, 28, 32, 36, or 40 carbon atoms (e.g., where at least one of which is a carbonyl carbon C(═O) in the ester group C(═O)O).

In Formula I, B can include one or both of glycolic and lactic units. When B contains both glycolic and lactic units, the units in the oligomeric residue can have a block or random arrangement. The total number n of glycolic/lactic units in B can be at least 4, 5, 6, 7, 8, 9, 10, 12, or 14 and/or up to 5, 6, 8, 10, 12, 14, 16, 18, 20, 30, 40, or 50.

In Formula I, when C is not an OH group, C can generally include the same options as A, but it can include comparatively shorter hydrocarbon segments as compared to A (e.g., when A is long enough to substantially provide the desired waxy character). In addition to the carboxylic and unsaturated functional groups that are possible for A, C can also include an additional oligomeric residue of glycolic/lactic units along its length (i.e., analogous to and in addition to B). For example, the C group can include at least 2, 3, 4, 6, 8, 10, 12, 14, 16, 18, 20, or 24 and/or up to 4, 6, 8, 12, 16, 20, 24, 28, 32, 36, or 40 carbon atoms (e.g., where at least one of which is a carbonyl carbon C(═O) in the ester group C(═O)O).

In Formula I, the linking groups a and b, when present, can include residues of diols or polyols, for example condensation reaction products linking carboxylic functional groups in A, B, and/or C via resulting ester linkages (e.g., via resulting alkylene groups). Both linking groups need not be present, since a terminal hydroxy functional group the oligomeric B group can react directly with a terminal carboxylic group in precursor reactants forming the A and/or C groups.

In various refinements of Formula I, either or both of linkers a and b can be present or absent (e.g., only a present, only b present, both a and b present). For example, linker a can be present when a diol linker is used to react with carboxylic terminal groups in A and B. Similarly, linker b can be present when a diol linker is used to react with carboxylic terminal groups in B and C.

In another aspect, the disclosure relates to a synthetic wax according to the following Formula IA and/or IB (e.g., alone or in admixture): R¹C(═O)O—R²—[—OC(═O)—CHR₃—]ₙ—O—R⁴ (IA); and/or R¹C(═O)O—[—CHR₃—OC(═O)—]ₙ—R²—O—R⁴ (IB). In Formulas IA and IB, R¹ is a hydrocarbon group having 11 to 39 carbon atoms; R² is a hydrocarbon linking group having 2 to 18 carbon atoms; n is 4 to 20 (or 4 to 50, or 6 to 14); R³ is independently H (i.e., glycolic acid residue) or CH₃ (i.e., lactic acid residue) for each of the n repeat units (i.e., where one or both of glycolic acid and lactic acid units can be included in the n repeat units); and R⁴ is H or hydrocarbon group having 2 to 40 carbon atoms, with the proviso that when R⁴ is H, then R¹ contains at least one of a carboxylic group and an unsaturated carbon-carbon double bond.

Formulas IA and IB generally represent a more specific structure of Formula I. R¹ in combination with the adjacent ester group represents specific selections for A. R² represents specific selections for embodiments when linker a is present (Formula IA) or when linker b is present (Formula IB). The n glycolic/lactic repeat units represent the oligomeric residue B, with the orientation/directionality of the repeat units depending on the location/presence of R² linking groups. R⁴ in combination with the adjacent oxygen atom represents specific selections for B (e.g., where R⁴ being H corresponds to C being OH).

R¹ can include linear, branched, or cyclic, saturated or unsaturated, substituted or unsubstituted hydrocarbon groups. For example, R¹ can include linear, branched, or cyclic, substituted or unsubstituted alkyl or alkenyl (e.g., having one or more C═C unsaturated bonds) groups. The R¹ group can be substituted with or otherwise include a carboxylic group (e.g., in acid or salt form) or an unsaturated (polymerizable) C═C unsaturated bond. The relatively long hydrocarbon character of R¹ imparts at least some of the waxy character to the synthetic wax. The R¹ group can include at least 8, 11, 13, 15, 17, 19, or 23 and/or up to 15, 19, 23, 27, 31, 35, or 39 carbon atoms. In embodiments, the R¹ group can include hydrocarbon groups of multiple different lengths, carbon atoms, etc., such as when the synthetic wax is formed using a mixture or blend of fatty acids, which can be a pre-selected blend or a naturally resulting blend from natural source of fats, oils, fatty acid (tri)glycerides, etc. In embodiments, the R¹ group can include a C17 alkyl group (e.g., representing a stearic acid residue or stearate ester in combination with the adjacent carboxylate group), a C13 alkyl group (e.g., myristic acid/ester), a C15 alkyl group (e.g., palmitic acid/ester), a C19 alkyl group (e.g., arachidic acid/ester), a C21 alkyl group (e.g., behenic acid/ester), and combinations thereof (e.g., a synthetic wax formed from a mixture or blend of fatty acids).

R² can include linear, branched, or cyclic, saturated or unsaturated, substituted or unsubstituted hydrocarbon groups. For example, R² can include linear, branched, or cyclic, substituted or unsubstituted alkyl or alkenyl (e.g., having one or more C═C unsaturated bonds) groups. The R² group generally represents an ester condensation product between a diol or polyol as a linker between a fatty acid, glycolic acid, and/or lactic acid as the corresponding R¹/R⁴ groups (or A/C groups) and the corresponding oligomeric residue (or B group). In embodiments, the R² group can include a C2 to C18 alkylene or hydroxyalkylene group, for example where a C2 alkylene group represents an ethylene glycol residue, a C3 alkylene group represents a propylene glycol residue, a C3 hydroxyalkyl group represents a glycerin residue, etc. The R² group can include at least 2, 3, 4, 5, or 6 and/or up to 3, 4, 5, 6, 8, or 18 carbon atoms.

As described above for B, the n glycolic/lactic repeat units form an oligomeric residue that facilitates degradation under mild conditions while still providing a thermally stable/thermally processable synthetic wax material. The oligomeric residue can include one or both of glycolic and lactic units. When the oligomeric residue contains both glycolic and lactic units, the units in the oligomeric residue can have a block or random arrangement. The total number n of glycolic/lactic units in B can be at least 4, 5, 6, 7, 8, 9, 10, 12, or 14 and/or up to 5, 6, 8, 10, 12, 14, 16, 18, 20, 30, 40, or 50. The orientation/direction of the glycolic/lactic repeat units depends on whether a linking group is included in the synthetic wax and/or the time during the synthesis process at which the glycolic/lactic units are reacted with the corresponding reactants forming the R¹, R¹ (when present), and R⁴ groups.

When R⁴ is not an H atom, R⁴ can generally include the same options as described above for C or as described above for R¹ when R⁴ includes a carbonyl carbon C(═O), but R⁴ can include comparatively shorter hydrocarbon segments as compared to A or R¹ (e.g., when A or R¹ is long enough to substantially provide the desired waxy character). In addition to the carboxylic and unsaturated functional groups that are possible for R⁴, R⁴ can also include carbonyl carbon (i.e., forming an ester group with the adjacent oxygen atom) and/or an additional oligomeric residue of glycolic/lactic units along its length (i.e., analogous to and in addition to B in Formula I or the n-glycolic/lactic oligomeric segment in Formulas IA/IB). For example, the R⁴ group can include at least 2, 3, 4, 6, 8, 10, 12, 14, 16, 18, 20, or 24 and/or up to 4, 6, 8, 12, 16, 20, 24, 28, 32, 36, or 40 carbon atoms, such where a terminal carbon atom is a carbonyl carbon at the bonding location with the oxygen atom to provide an ester group. In embodiments and similar to R¹ above, the R⁴ group can include hydrocarbon groups of multiple different lengths, carbon atoms, etc., such as when the synthetic wax is formed using a mixture or blend of fatty acids.

In a refinement of Formula IA and/or IB, R¹ is C₁₇H₃₅ (i.e., a stearic fatty acid residue); R² is C₂H₄ (i.e., an ethylene glycol linker); and/or OR⁴ is OC(═O)—C₁₇H₃₅ (i.e., a stearic fatty acid residue).

In a refinement of Formula IA and/or IB, R³ is H for all n repeat units (i.e., only glycolic acid residues).

In a refinement of Formula IA and/or IB, R³ is CH₃ for all n repeat units (i.e., only lactic acid residues).

In a refinement of Formula IA and/or IB, R¹ is selected from the group consisting of: (i) R^{1A} (e.g., saturated or unsaturated fatty acid ester group), and (ii) R^{1A}-d- (e.g., saturated or unsaturated fatty acid ester group with a linker group d containing a pendant carboxylic or carboxylate group in acid or salt form, such as an alkali or other metal salt (e.g., Na, K, Ca, Mg, Zn), ammonium salt, etc.). R^{1A} is a C₈ to C₃₉ alkyl group or unsaturated analog thereof (e.g., an alkenyl group, dienyl group, or trienyl group with 1, 2, or 3 unsaturated C═C groups along the length of an otherwise saturated alkyl group) More generally, the R^{1A} group can include at least 8, 11, 13, 15, 17, 19, or 23 and/or up to 15, 19, 23, 27, 31, 35, or 39 carbon atoms, for example with 0, 1, 2, or 3 unsaturated C═C groups. The d group is a hydrocarbon linking group containing 1 to 4 carbon atoms (e.g., 1, 2, 3, or 4 carbon atoms) and a pendant carboxylic acid group or salt thereof. For example, the linker d can be represented by *—CH(COOH)-# (1-carbon linking group with pendant, terminal carboxylic group), *—CH(COOH)—CH₂-# (2-carbon linking group with pendant, terminal carboxylic group), *—CH₂—CH(COOH)-# (2-carbon linking group with pendant, terminal carboxylic group), or *—CH(CH₂COOH)-# (2-carbon linking group with pendant, terminal carboxylic group with an intervening methylene group), where “*” is the point of attachment/bonding to R^{1A}, and “#” is the point of attachment/bonding to the carbonyl carbon/ester group adjacent to R¹ in Formula IA or IB. The illustrated COOH group in the foregoing structure for the linker d can be in acid form (as shown) or in salt form, such as an alkali or other metal salt (e.g., Na, K, Ca, Mg, Zn), ammonium salt, etc. In some embodiments, R¹ (or A in Formula I) or its substituents can be substituted with one or more other functional groups such as epoxy groups, halogens, ether groups, acetal groups, etc.

In a particular refinement, R¹ is R^{1A}, and R^{1A} is the C₁₁ to C₃₉ alkyl group (i.e., saturated/not unsaturated). In this case, a saturated fatty acid ester is used to provide the waxy character of the wax via the A or R¹ group. In an alternative refinement, R^{1A} can include an unsaturated group, for example to provide a means for curing, such as by addition of a peroxide (e.g., dicumyl peroxide) initiator to the wax. Such curing or crosslinking can make the resulting wax more difficult to melt, thus increasing its thermal resistance.

In a particular refinement, R¹ is R^{1A}-d- or the salt thereof. This represents an alternative embodiment in which a pendant carboxylic/carboxylate group provides some hydrophilic character for improved dispersion and/or dissolution in water, such as when degrading the wax, forming a coating with the wax, etc. More specifically, the carboxylic groups provide several functions. (1) The carboxylic groups provide easy removal from coated paper by increasing pH above 7. (2) The carboxylic groups can be used to emulsify the wax in a basic liquid medium (such as ammonia), the wax is then applied as a coating from the basic medium, and then gentle heat is applied to remove the liquid medium (as well as ammonia), leaving the wax as the coating. This provides a water-repellent coating, which can be simply removed from the surface by neutralizing the wax/exposing it to a basic medium again. (3) The carboxylic groups permit complexation with divalent or trivalent metal cations, for example Ca⁺². Such complexation can offer better water resistance as it can form synthetic wax dimers (e.g., when using a divalent cation) or trimers (e.g., when using a trivalent cation). As above, the R^{1A} group can be saturated to provide a more crystalline solid wax. Alternatively, the R^{1A} group can be an unsaturated chain to provide a means for curing as described above, for example when an alkenyl-substituted anhydride, such as a dodecenyl succinic anhydride, is used to provide the waxy character of the wax via the A or R¹ group.

In a refinement of Formula IA and/or IB, OR⁴ is selected from the group consisting of: (i) —OC(═O)—R⁵ (e.g., saturated or unsaturated fatty acid ester group), (ii) —OC(═O)-d-R⁵ or a salt thereof (e.g., saturated or unsaturated fatty acid ester group with a linker group d containing a pendant carboxylic or carboxylate group in acid or salt form, such as an alkali metal salt (e.g., Na, K), ammonium salt, etc.), (iii) —OC(═O)—CH═CH₂ (e.g., acrylate group), (iv) —OC(═O)—C(CH₃)═CH₂ (e.g., methacrylate group), (v) —OC(═O)—C(COOH)═CH₂ (e.g., itaconic group), (vi) —[—OC(═O)—CHR₃—]ₘ—OC(═O)—R⁵ in the synthetic wax according to Formula IB (e.g., saturated or unsaturated fatty acid ester group as above, but attached via a second oligomeric glycolic/lactic residue), (vii) —[—OC(═O)—CHR₃—]ₘ—OC(═O)-d-R⁵ or a salt thereof in the synthetic wax according to Formula IB (e.g., saturated or unsaturated fatty acid ester group with a linker group d containing a pendant carboxylic or carboxylate group in acid or salt form as above, but attached via a second oligomeric glycolic/lactic residue), and (viii) combinations thereof (e.g., where different selections for OR⁴, R⁵ etc. can represent a blend of Formula IA and/or Formula IB synthetic waxes). R⁵ is a C₁ to C₃₉ alkyl group or unsaturated analog thereof (e.g., an alkenyl group, dienyl group, or trienyl group with 1, 2, or 3 unsaturated C═C groups along the length of an otherwise saturated alkyl group). More generally, the R⁵ group can include at least 1, 2, 3, 5, 7, 9, 11, 13, 15, 17, 19, or 23 and/or up to 3, 5, 7, 11, 15, 19, 23, 27, 31, 35, or 39 carbon atoms, for example with 0, 1, 2, or 3 unsaturated C═C groups. The d group is a hydrocarbon linking group containing 1 to 4 carbon atoms (e.g., 1, 2, 3, or 4 carbon atoms) and a pendant carboxylic acid group or salt thereof, with the same options as described above (e.g., where “#” is the point of attachment/bonding to R⁵, and “*” is the point of attachment/bonding to the adjacent carbonyl carbon/ester group). The index m is 4 to 20 or 4 to 50, and it more generally can have the same options for selections and subranges from 4 to 20 or 4 to 50 as described above for the index n. In some embodiments, OR⁴ (or C in Formula I) or its substituents can be substituted with one or more other functional groups such as epoxy groups, halogens, ether groups, acetal groups, etc.

In a particular refinement, OR⁴ is —OC(═O)—R⁵, and R⁵ is the C₁ to C₃₉ alkyl group (i.e., saturated/not unsaturated). In this case, a saturated fatty acid ester is used to provide additional waxy character of the wax via the C or R⁵ group. In an alternative refinement, R⁵ can include an unsaturated group, for example to provide a means for curing, such as by addition of a peroxide (e.g., dicumyl peroxide) initiator to the wax. Such curing or crosslinking can make the resulting wax more difficult to melt, thus increasing its thermal resistance.

In a particular refinement, OR⁴ is —OC(═O)-d-R⁵ or a salt thereof. As above, this represents an alternative embodiment in which a pendant carboxylic/carboxylate group provides some hydrophilic character for improved dispersion and/or dissolution in water, such as when degrading the wax, forming a coating with the wax, etc., in particular facilitating coating application/removal in a basic medium and synthetic wax complexation with metal cations.

In a particular refinement, OR⁴ is —[—OC(═O)—CHR₃—]ₘ—OC(═O)—R⁵ or —[—OC(═O)—CHR₃—]ₘ—OC(═O)-d-R⁵ or a salt thereof. This represents an embodiment in which an additional oligomeric glycolic/lactic residue is incorporated to provide improved emulsification characteristics, for example for comparatively larger-MW synthetic waxes.

In a particular refinement, OR⁴ is —OC(═O)—CH═CH₂ or —OC(═O)—C(CH₃)═CH₂. This represents an embodiment incorporating a (meth)acrylate group that can be cured in a coating once applied to a substrate or polymerized first in a solvent, water, or melt-phase, and then used for coating. The cured or polymerized waxes offer excellent thermal sealing properties and high melting points (Tm) suitable for hot food/beverage applications.

In a refinement of Formula IA and/or IB, wherein R² is a glycerin residue comprising a pendant group selected from (i) OH, (ii) —OC(═O)—R⁶ (e.g., saturated or unsaturated fatty acid ester group, such as from original triglyceride), and (iii) —[—OC(═O)—CHR₃—]ₒ—OC(═O)—R⁶ (e.g., saturated or unsaturated fatty acid ester group as above, but attached via an additional oligomeric glycolic/lactic residue). R⁶ is a C₁ to C₃₉ alkyl group or unsaturated analog thereof (e.g., an alkenyl group, dienyl group, or trienyl group with 1, 2, or 3 unsaturated C═C groups along the length of an otherwise saturated alkyl group). More generally, the R⁵ group can include at least 1, 2, 3, 5, 7, 9, 11, 13, 15, 17, 19, or 23 and/or up to 3, 5, 7, 11, 15, 19, 23, 27, 31, 35, or 39 carbon atoms, for example with 0, 1, 2, or 3 unsaturated C═C groups. The index o is 4 to 20 or 4 to 50, and it more generally can have the same options for selections and subranges from 4 to 20 or 4 to 50 as described above for the index n. In some embodiments, it is possible to start with a fatty acid triglyceride as a source material for forming the synthetic wax, and then perform one or more transesterification reaction(s) to insert one or more glycolic/lactic oligomeric segments between the glycerin backbone and pendant fatty acid structures to arrive at synthetic wax structures having 1, 2, or 3 fatty acid segments attached to a glycerin backbone with 1, 2, or 3 intervening glycolic/lactic oligomeric segments between the fatty acid segments and the glycolic/lactic oligomeric segments. This could be represented, for example, in the context of Formula IA or IB with R² as a glycerin residue with an additional pendant fatty acid ester group, with or without an additional inserted glycolic/lactic oligomeric segment.

In a refinement, the synthetic wax comprises at least one synthetic wax according to Formula IA (e.g., only one or more waxes according to Formula IA and no waxes according to Formula IB, such as where there is a blend of Formula IA waxes with different selections for one or more of R¹, R², R³, R⁴, and/or n).

In a refinement, the synthetic wax comprises at least one synthetic wax according to Formula IB (e.g., only one or more waxes according to Formula IB and no waxes according to Formula IA, such as where there is a blend of Formula IB waxes with different selections for one or more of R¹, R², R³, R⁴, and/or n).

In a refinement, the synthetic wax comprises at least one synthetic wax according to Formula IA; and at least one synthetic wax according to Formula IB (e.g., a blend of one or more waxes according to Formula IA and one or more waxes according to Formula IB, such as with the same or different selections for one or more of R¹, R², R³, R⁴, and/or n).

Various refinements of the disclosed synthetic wax compositions in any of their aspects are possible (e.g., according to any of Formulas I, IA, or IB).

In a refinement, the synthetic wax can have a melting temperature in a range of 30° C. to 160° C. or 30° C. to 90° C. Generally, lower melting temperatures are characteristic of unpolymerized, uncrosslinked, uncured, and/or uncomplexed embodiments as described above. Similarly, higher melting temperatures are characteristic of polymerized, crosslinked, cured, and/or complexed (e.g., with metal divalent or trivalent cations) embodiments as described herein. For example, the melting temperature can be at least 30, 40, 50, 60, 70, 80, 90, 100, or 110° C. and/or up to 40, 50, 60, 70, 80, 90, 100, 110, 120, 130, 140, 150, or 160° C.

In a refinement, the synthetic wax can have a molecular weight in a range of 2000-100000 g/mol or 300-5000 g/mol. Generally, lower molecular weights are characteristic of unpolymerized, uncrosslinked, uncured, and/or uncomplexed embodiments as described above. Similarly, higher molecular weights are characteristic of polymerized, crosslinked, cured, and/or complexed (e.g., with metal divalent or trivalent cations) embodiments as described above. For example, the molecular weight can be at least 300, 500, 700, 1000, 2000, 3000, 5000, 10000, 20000, 30000, or 50000 g/mol and/or up to 800, 1000, 2000, 3000, 5000, 10000, 20000, 30000, 50000, or 100000 g/mol. The foregoing weights can represent the molecular weights of specific synthetic wax molecules or an average (e.g., number- or weight-average) molecular weight for a polymeric, cured, crosslinked, or complexed wax containing a distribution of different sizes/molecular weights.

In another aspect, the disclosure relates to a high-melting synthetic wax comprising a polymerized and/or a metal-complexed synthetic wax according to the disclosure. For example, a high-melting synthetic wax can comprise a polymerization reaction product of a synthetic wax according to Formula I in which at least one (or only one) of A and C contains at least one (or only one) unsaturated carbon-carbon (polymerizable) double bond. Alternatively, a high-melting synthetic wax can comprise a polymerization reaction product of a synthetic wax according to Formula IA and/or IB in which at least one (or only one) of R¹ and R⁴ contains at least one (or only one) carbon-carbon (polymerizable) double bond. Alternatively, a high-melting synthetic wax can comprise a complex between a polyvalent metal cation (e.g., divalent or trivalent) and a synthetic wax according to Formula I in which at least one (or only one) of A and C contains at least one (or only one) carboxylic group. Alternatively, a high-melting synthetic wax can comprise a complex between a polyvalent metal cation (e.g., divalent or trivalent) and a synthetic wax according to Formula IA and/or IB in which at least one (or only one) of R¹ and R⁴ contains at least one (or only one) carboxylic group.

In another aspect, the disclosure relates to a synthetic wax composition comprising the synthetic wax according to any of the variously disclosed aspects, refinements, embodiments, etc.; and one or more additives blended with the synthetic wax.

In a refinement, the additives are present in an amount of 1 wt. % to 50 wt. % or 5 wt. % to 20 wt. % relative to the synthetic wax composition. More generally, the additives can be present in an amount of at least 0.1, 1, 2, 3, 5, 7, 10, 12, 15, or 20 wt. % and/or up to 1, 2, 4, 6, 8, 10, 12, 16, 20, 25, 30, 40, or 50 wt. %, where the foregoing amounts can apply independently to individual additives and/or all additive combined. The additives are not particularly limited and can include fibers, particles, etc. For example, the additives can be selected from the group consisting of inorganic fillers (e.g., silica, calcium carbonate, titanium dioxide), polymeric fillers (e.g., polyvinyl acetate, polymerized waxes, biodegradable polymers, such as biodegradable polymers including carboxylic and/or hydroxyl functional groups), nanoparticles (e.g., cellulose nanocrystals/cellulose nanofibrils (functionalized and non-functionalized), carbon nanotubes, graphene oxides (functionalized and non-functionalized), clays or nanoclays (functionalized and non-functionalized)), natural waxes (e.g., carnauba), plasticizers (e.g., hydrophobic or hydrophilic), and combinations thereof.

In another aspect, the disclosure relates to a synthetic wax dispersion comprising an aqueous medium (e.g., water alone or in combination with other solvents or solutes); and a synthetic wax according to any of the variously disclosed aspects, refinements, embodiments, etc. dispersed in the aqueous medium. Such waterborne or other aqueous dispersions can be used to apply a coating on a paper or other substrate. For example, the synthetic wax can be dispersed in hot water, and then the hot water dispersion can be applied onto paper or other another substrate to form the wax coating (e.g., after drying/water evaporation).

In another aspect, the disclosure relates to a coated article comprising: a substrate; and a coating on the substrate, the coating comprising the synthetic wax or the synthetic wax composition according to any of the variously disclosed aspects, refinements, embodiments, etc. As described above, the coating can be applied from an aqueous dispersion or emulsion of the synthetic wax. In other embodiments, the coating can be applied in the form of a melt coating of the synthetic wax (e.g., extrusion and non-extrusion melt-coating). The substrate is suitably a cellulosic substrate such as a paper substrate. These synthetic wax can be used as a coating directly on a cellulosic substrate, or as a top layer on an already-coated cellulosic substrate (e.g., with a first coating on the substrate such as a polymer like starch, and then a top or second coating of the synthetic wax). The synthetic wax can also be applied as a coating on other materials such as plastic films/bottles; water repellent fabrics; automotive polishes, as mold release agents in mold making, and other non-packaging applications. Typical coating thicknesses can be 1 μm to 1000 μm or 20 μm to 50 μm.

In a refinement, the synthetic wax coating is applied as a second layer on top of a pre-coated paper. The pre-coated paper is obtained by coating a paper substrate with a hydrophilic polymer or a blend including a hydrophilic polymer, for example including (i) a hydrophilic polymer (e.g., 80 or 90 to 95, 98, or 99.8 wt. % hydrophilic polymer relative to blend), (ii) 0.1-10 wt. % polyethylene imine or other amine/imine polymer different from the hydrophilic polymer, and/or (iii) 0.1-10 wt. % carbonate salt (e.g., sodium carbonate, calcium carbonate, etc.). Once the hydrophilic polymer or blend thereof is coated on paper as first layer via melt-coating, solvent coating, aqueous coating, or other suitable method, then the synthetic wax coating can be applied as a second layer thereon. The pre-coating can improve recycling during paper repulping by more rapidly degrading the waxes, and/or improve barrier performance.

In a refinement, the coated article has a kit rating in a range of 4 to 12; and/or the coated article has a cobb (or cobb1800) rating of 20 g/m² or less. For example, the kit rating can be at least 4, 5, 6, 7, 8, 9, 10, or 11 and/or up to 8, 9, 10, 11, or 12. Alternatively or additionally, the cobb rating (or cobb1800 rating) can be at least 0.1, 0.2, 0.5, 1, 2, 3, 6, 8, 10, 12, or 15 g/m² and/or up to 3, 5, 7, 10, 15, or 20 g/m². Suitable methods for determining the ratings include TAPPI methods T599 μm-96 (kit), UM 557 (kit), and T441 om-09 (cobb).

In a refinement, the coating on the coated article has at least one of properties (I), (II), and (III): (I) the coated article has a relative permeability for water vapor of 0.5 or less, relative to a corresponding substrate without the synthetic wax coating thereon; (II) the coated article has a water contact angle in a range of 80° to 120° for a 10 μL deionized water droplet measured 30 sec after application of the droplet; and (III) the coated article has an oil contact angle in a range of 40° to 75° for a 10 μL castor oil droplet measured 30 sec after application of the droplet.

In another aspect, the disclosure relates to a method for degrading a synthetic wax, the method comprising: contacting the synthetic wax or synthetic wax composition according to any of the variously disclosed aspects, refinements, embodiments, etc. with at least one of a carbonate salt, a bicarbonate salt, and an aqueous solution thereof at a temperature and for a time sufficient to degrade the synthetic wax.

The synthetic wax is degradable under mild conditions, for example by contact with carbonate/bicarbonate salts or immersion in aqueous carbonate/bicarbonate solutions (e.g., 5-50 wt. % or 1-50 wt. % (bi)carbonate or (bi)carbonate salt in water, such as about 1, 2, 3, 5, 10, 15, 20, 30, 40, or 50 wt. %. Suitable (bi)carbonate salts usable as is or to form corresponding carbonate ions in solution (CO₃²⁻) can include alkali metal carbonates (e.g., sodium carbonate, sodium bicarbonate), ammonium carbonate, ammonium bicarbonate, etc. Degradation also can be effected in a mild acidic medium (e.g., pH 4-6 aqueous medium) or a mild basic medium (e.g., pH 8-10 aqueous medium). Degradation generally includes cleaving one or more glycolic/lactic ester bonds in the glycolic/lactic oligomeric residue in the original synthetic wax molecule, breaking it into smaller fragments, which can facilitate removal and/or separation of a wax coating from its substrate. Degradation temperatures are suitably elevated related to ambient conditions, but need not be excessively high. For example, degradation temperature can be in a range of 20-95° C., 40-95° C., 40-80° C., or 60-80° C. Degradation times can range from about 0.1-5 hr (or 0.2-2 hr).

The synthetic wax can also be in the form of synthetic wax composition blended with other additives, for example a coating on a substrate in a method for removing/degrading the coating. For example, when the coating is on a paper substrate, the method for degrading the synthetic wax can be part of a recycling method to remove the wax coating, followed by repulping and recycling the paper. Typically, the degraded wax is in the form of smaller hydrocarbons relative to the original synthetic wax, often with carboxylic groups such as from stearic acid or pendant carboxylic groups, which in a basic medium makes the degraded wax water soluble and/or water-emulsifiable, which in turn facilitates removal, separation, and/or recovery of the wax material from the substrate. In some cases, emulsification can occur upon neutralization of a synthetic wax containing a carboxylic group in its normal form (e.g., prior to degradation).

In some embodiments, the synthetic wax can be recovered either in addition or as an alternative to the degradation described above. For example, when the synthetic wax includes pendant carboxylic groups (e.g., an emulsifiable synthetic wax), removal of the synthetic wax coating is possible by neutralization in an aqueous medium to emulsify and remove the synthetic wax from its substrate. Such an emulsified wax can be recovered and reverted back to a solid by changing pH, for example for use in subsequent coating step (e.g., in an overall recycling process).

While the disclosed articles, apparatus, methods, and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### BRIEF DESCRIPTION OF THE DRAWINGS

For a more complete understanding of the disclosure, reference should be made to the following detailed description and accompanying drawings wherein:

FIG. 1 is a scheme illustrating steps for forming a synthetic wax according to an embodiment of the disclosure.

FIG. 2 is a scheme illustrating steps for forming a synthetic wax according to another embodiment of the disclosure.

FIG. 3 is a scheme illustrating steps for forming a synthetic wax according to another embodiment of the disclosure.

FIG. 4 is a scheme illustrating steps for forming a synthetic wax according to another embodiment of the disclosure.

FIG. 5 is a scheme illustrating steps for forming a synthetic wax according to another embodiment of the disclosure.

FIG. 6 illustrates a chemical structure for a synthetic wax according to another embodiment of the disclosure.

FIG. 7 is a scheme illustrating steps for forming a synthetic wax according to another embodiment of the disclosure.

FIG. 8 is a scheme illustrating steps for forming a synthetic wax according to another embodiment of the disclosure.

FIG. 9 is a scheme illustrating steps for forming a synthetic wax according to another embodiment of the disclosure.

FIG. 10 is a scheme illustrating steps for forming a synthetic wax according to another embodiment of the disclosure.

FIG. 11 is a schematic of a coated article including a synthetic wax coating according to the disclosure.

FIG. 12 is a diagram illustrating measurement of a contact angle for a liquid droplet on a surface.

### DETAILED DESCRIPTION

The disclosure relates to synthetic wax compositions including at least one long-chain hydrocarbon residue having 12 to 40 carbon atoms, and at least one oligomeric residue containing 4 to 20 or 4 to 50 glycolic acid units and/or lactic acid units. The synthetic wax can include other functional groups such as pendant carboxylic groups to facilitate emulsification and/or metal complexation of the wax, and/or unsaturated carbon-carbon double bonds to facilitate curing or crosslinking of the wax. The synthetic wax can be used as a coating on a variety of substrates, for example paper substrates, to impart water and/or oil resistance to the substrate. The presence of the oligomeric glycolic/lactic residue in the wax permits chemical degradation under very mild conditions, which in turn facilitates removal, repulping, and/or recycling of the synthetic wax.

The disclosed synthetic, biodegradable wax is an alternative to natural wax. The synthetic wax has performance characteristics, such as water and oil resistance, matching that of a natural wax like beeswax and carnauba wax. The synthetic wax developed can include degradable links, for example glycolic acid units and/or lactic acid units, that impart to the waxes universal compostabilty (e.g., biodegrade in the ocean, lake, or other water environment, soil, and industrial compost environment), as well as on-demand degradability during repulping (e.g., washing or otherwise being easily removed from paper, etc.). The biodegradability aspect of the synthetic wax mitigates the accumulation of microplastics that are otherwise building up in the ocean and soil, while repulping enables the synthetic wax-coated paper to stay in use for repetitive use. The synthetic wax can provide excellent water resistance to a coated paper substrate (e.g., Cobb1800 value of 10 g/m² or less, or 15 g/m² or less). The synthetic wax can be formulated with a desired melting point based on its intended application. For low-temperature applications, the synthetic wax can be formulated with a melting temperature (Tm) of about 70° C. or less. For high-temperature applications, the synthetic wax can be formulated with a melting temperature (Tm) up to about 140° C.

### Synthetic Wax

The synthetic wax according to the disclosure can be represented by the following Formula I:

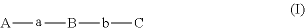

In Formula I, A can be a hydrocarbon ester group having 12 to 40 carbon atoms. A can include linear, branched, or cyclic, saturated or unsaturated, substituted or unsubstituted hydrocarbon groups having at least one ester functional group. For example, A can include linear, branched, or cyclic, substituted or unsubstituted alkyl or alkenyl (e.g., having one or more C═C unsaturated bonds) groups with a terminal ester functional group at the point of bonding to a or B. The A group can be substituted with or otherwise include a carboxylic group (e.g., in acid or salt form) or an unsaturated (polymerizable) C═C unsaturated bond. The relatively long hydrocarbon character of A imparts at least some of the waxy character to the synthetic wax. The A group can include at least 8, 12, 14, 16, 18, 20, or 24 and/or up to 16, 20, 24, 28, 32, 36, or 40 carbon atoms (e.g., where at least one of which is a carbonyl carbon C(═O) in the ester group C(═O)O).

In Formula I, B can be an oligomeric residue of (i) n glycolic acid units, (ii) n lactic acid units, or (iii) n glycolic acid units and lactic acid units in total, where n is 4 to 50, 4 to 20, or 6 to 14. B can include one or both of glycolic and lactic units. When B contains both glycolic and lactic units, the units in the oligomeric residue can have a block or random arrangement. The total number n of glycolic/lactic units in B can be at least 4, 5, 6, 7, 8, 9, 10, 12, or 14 and/or up to 5, 6, 8, 10, 12, 14, 16, 18, 20, 30, 40, or 50.

In Formula I, C can be either OH or a hydrocarbon ester group having 2 to 40 carbon atoms, with the proviso that when C is OH, then A contains at least one of a carboxylic group and an unsaturated carbon-carbon double bond. When C is not an OH group, C can generally include the same options as A, but it can include comparatively shorter hydrocarbon segments as compared to A (e.g., when A is long enough to substantially provide the desired waxy character). In addition to the carboxylic and unsaturated functional groups that are possible for A, C can also include an additional oligomeric residue of glycolic/lactic units along its length (i.e., analogous to and in addition to B). For example, the C group can include at least 2, 3, 4, 6, 8, 10, 12, 14, 16, 18, 20, or 24 and/or up to 4, 6, 8, 12, 16, 20, 24, 28, 32, 36, or 40 carbon atoms (e.g., where at least one of which is a carbonyl carbon C(═O) in the ester group C(═O)O).

In Formula I, a and b are linking groups that can be either present or absent (e.g., only a present, only b present, both a and b present). When present, a is a linking group between A and B. When present, and b is a linking group between B and C. Each of the linking groups a and b, when present, can include residues of diols or polyols, for example condensation reaction products linking carboxylic functional groups in A, B, and/or C via resulting ester linkages (e.g., via resulting alkylene groups). Both linking groups need not be present, since a terminal hydroxy functional group the oligomeric B group can react directly with a terminal carboxylic group in precursor reactants forming the A and/or C groups. For example, linker a can be present when a diol linker is used to react with carboxylic terminal groups in A and B. Similarly, linker b can be present when a diol linker is used to react with carboxylic terminal groups in B and C.

The synthetic wax according to the disclosure additionally or alternatively can be represented by the following Formula IA and/or IB, for example alone (e.g., only wax(es) of Formula IA) or in admixture (e.g., wax(es) of Formula IA and wax(es) of Formula IB):

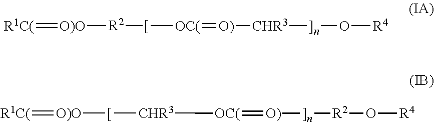

Formulas IA and IB generally represent a more specific structure of Formula I. R¹ in combination with the adjacent ester group represents specific selections for A. R² represents specific selections for embodiments when linker a is present (Formula IA) or when linker b is present (Formula IB). The n glycolic/lactic repeat units represent the oligomeric residue B, with the orientation/directionality of the repeat units depending on the location/presence of R² linking groups. R⁴ in combination with the adjacent oxygen atom represents specific selections for B (e.g., where R⁴ being H corresponds to C being OH).

In Formulas IA and IB, R¹ can be a hydrocarbon group having 11 to 39 carbon atoms. R¹ can include linear, branched, or cyclic, saturated or unsaturated, substituted or unsubstituted hydrocarbon groups. For example, R¹ can include linear, branched, or cyclic, substituted or unsubstituted alkyl or alkenyl (e.g., having one or more C═C unsaturated bonds) groups. The R¹ group can be substituted with or otherwise include a carboxylic group (e.g., in acid or salt form) or an unsaturated (polymerizable) C═C unsaturated bond. The relatively long hydrocarbon character of R¹ imparts at least some of the waxy character to the synthetic wax. The R¹ group can include at least 8, 11, 13, 15, 17, 19, or 23 and/or up to 15, 19, 23, 27, 31, 35, or 39 carbon atoms. In embodiments, the R¹ group can include hydrocarbon groups of multiple different lengths, carbon atoms, etc., such as when the synthetic wax is formed using a mixture or blend of fatty acids, which can be a pre-selected blend or a naturally resulting blend from natural source of fats, oils, fatty acid (tri)glycerides, etc. In embodiments, the R¹ group can include a C17 alkyl group (e.g., representing a stearic acid residue or stearate ester in combination with the adjacent carboxylate group), a C13 alkyl group (e.g., myristic acid/ester), a C15 alkyl group (e.g., palmitic acid/ester), a C19 alkyl group (e.g., arachidic acid/ester), a C21 alkyl group (e.g., behenic acid/ester), and combinations thereof (e.g., a synthetic wax formed from a mixture or blend of fatty acids).

In Formulas IA and IB, R² can be a hydrocarbon linking group having 2 to 18 carbon atoms. R² can include linear, branched, or cyclic, saturated or unsaturated, substituted or unsubstituted hydrocarbon groups. For example, R² can include linear, branched, or cyclic, substituted or unsubstituted alkyl or alkenyl (e.g., having one or more C═C unsaturated bonds) groups. The R² group generally represents an ester condensation product between a diol or polyol as a linker between a fatty acid, glycolic acid, and/or lactic acid as the corresponding R¹/R⁴ groups (or A/C groups) and the corresponding oligomeric residue (or B group). In embodiments, the R² group can include a C2 to C18 alkylene or hydroxyalkylene group, for example where a C2 alkylene group represents an ethylene glycol residue, a C3 alkylene group represents a propylene glycol residue, a C3 hydroxyalkyl group represents a glycerin residue, etc. The R² group can include at least 2, 3, 4, 5, or 6 and/or up to 3, 4, 5, 6, 8, or 18 carbon atoms.

In Formulas IA and IB, n can be 4 to 50, 4 to 20, or 6 to 14. R³ can be independently H (i.e., glycolic acid residue) or CH₃ (i.e., lactic acid residue) for each of the n repeat units, such that one or both of glycolic acid and lactic acid units can be included in the n repeat units. The n glycolic/lactic repeat units form an oligomeric residue that facilitates degradation under mild condition while still providing a thermally stable/thermally processable synthetic wax material. The oligomeric residue can include one or both of glycolic and lactic units. When the oligomeric residue contains both glycolic and lactic units, the units in the oligomeric residue can have a block or random arrangement. The total number n of glycolic/lactic units in Formulas IA and IB can be at least 4, 5, 6, 7, 8, 9, 10, 12, or 14 and/or up to 5, 6, 8, 10, 12, 14, 16, 18, 20, 30, 40, or 50. The orientation/direction of the glycolic/lactic repeat units depends on whether a linking group is included in the synthetic wax and/or the time during the synthesis process at which the glycolic/lactic units are reacted with the corresponding reactants forming the R¹, R², and R⁴ groups.

In Formulas IA and IB, R⁴ can be H or hydrocarbon group having 2 to 40 carbon atoms, with the proviso that when R⁴ is H, then R¹ contains at least one of a carboxylic group and an unsaturated carbon-carbon double bond. When R⁴ is not an H atom, R⁴ can generally include the same options as described above for C or as described above for R¹ when R⁴ includes a carbonyl carbon C(═O), but R⁴ can include comparatively shorter hydrocarbon segments as compared to A or R¹ (e.g., when A or R¹ is long enough to substantially provide the desired waxy character). In addition to the carboxylic and unsaturated functional groups that are possible for R⁴, R⁴ can also include carbonyl carbon (i.e., forming an ester group with the adjacent oxygen atom) and/or an additional oligomeric residue of glycolic/lactic units along its length (i.e., analogous to and in addition to B in Formula I or the n-glycolic/lactic oligomeric segment in Formulas IA/IB). For example, the R⁴ group can include at least 2, 3, 4, 6, 8, 10, 12, 14, 16, 18, 20, or 24 and/or up to 4, 6, 8, 12, 16, 20, 24, 28, 32, 36, or 40 carbon atoms, such where a terminal carbon atom is a carbonyl carbon at the bonding location with the oxygen atom to provide an ester group. In embodiments and similar to R¹ above, the R⁴ group can include hydrocarbon groups of multiple different lengths, carbon atoms, etc., such as when the synthetic wax is formed using a mixture or blend of fatty acids.

In general, waxes according to Formulas I, IA, and IB can be synthesized using precursors that provide the structural units in the formulas after reaction (e.g., condensation reaction, ring-opening reaction). Suitable precursors for providing the A unit of Formula I can include carboxylic acids having a hydrocarbon chain having from 12 to 40 carbon atoms and esters of such acids. In particular, hydroxy-containing esters of such acids, such as hydroxyethyl esters. Similarly, precursors for providing the R¹C(═O)O— unit of Formula IA and Formula IB can include carboxylic acids having the formula R¹C(═O)OH and esters of such acids. Other suitable precursors for the A unit or the R¹C(═O)O— unit include succinic acid anhydrides having a hydrocarbon chain according to the definitions of A and R¹ in the respective formulas. As noted above, including an anhydride such as a succinic anhydride in the synthesis of the wax can introduce a pendant carboxylic or carboxylate group to the resulting wax. As described above, pendant carboxylic or carboxylate groups on the wax can provide several functions, including providing easy removal of the wax from coated paper by increasing pH above 7; enabling the wax to be emulsified in a basic medium and applied as a water-repellent coating which can be easily removed by re-exposure to a basic medium; and providing improved water resistance via complexation with multivalent cations.

The A unit of Formula I, and the R¹C(═O)O— unit of Formula IA and Formula IB, can be provided by a carboxylic acid having a hydrocarbon group corresponding to R¹.

Glycolide, lactide (e.g., L-lactide, DL-lactide, D-lactide, or blends thereof), or a mixture thereof can be the precursor for glycolic and/or lactic units comprising the synthetic wax, i.e., unit B of Formula I, the OC(═O)—CHR₃ unit of Formula IA, and the CHR₃—OC(═O) unit of Formula IB.

### Coated Article

As illustrated in FIG. 11, the disclosure provides a coated article 300 including a substrate 310 and a coating 320 on or adjacent to the substrate. The coating 320 can include the synthetic wax or the synthetic wax composition according to any of the variously disclosed embodiments. The coating can be applied by any suitable method, for example from an aqueous dispersion or emulsion of the synthetic wax (e.g., solvent casting). In other embodiments, the coating can be applied in the form of a melt coating of the synthetic wax (e.g., extrusion and non-extrusion melt-coating). In other embodiments, the coating can be applied by compression molding the synthetic wax (e.g., application of heat and pressure to a synthetic wax in contact with a substrate to be coated). The substrate 310 is suitably a cellulosic substrate such as a paper substrate. These synthetic wax can be used as a coating directly on a cellulosic substrate, or as a top layer on an already-coated cellulosic substrate (e.g., with a first coating on the substrate such as a polymer like starch, and then a top or second coating of the synthetic wax). The synthetic wax can also be applied as a coating on other materials such as plastic films/bottles; water repellent fabrics; automotive polishes, as mold release agents in mold making, and other non-packaging applications. Typical coating 320 thicknesses can be 1 μm to 1000 μm or 20 μm to 50 μm. For example, the coating 320 can have thickness of at least 1, 2, 5, 10, 20, 30, 50, 100, 200, or 300 μm and/or up to 30, 50, 70, 100, 200, 500, 700, or 1000 μm. Typical cast coatings can have thicknesses of 10 μm to 100 μm. As described herein, multiple coating layers can be applied to substrate 310 to form even thicker layers of the coating 320 (e.g., above 1000 μm, 2000 μm, or otherwise) if desired.

The coated article 300 of the disclosure includes a substrate 310. Examples of suitable substrates include, but are not limited to, porous substrates and other substrates. In the case of a coating on paper or other porous substrate, a layer or layers including polyethylene imine (PEI), polyacrylic acid (PAA), PEI-PAA, chitosan, starch, polyvinyl alcohol (PVOH), and/or blends thereof can be applied on the substrate as a first layer and then coated with the synthetic wax as a second layer (e.g., with the first layer positioned between and/or adhered to the substrate and the second layer). When the substrate 310 is a porous substrate, the coating 320 and/or the first layer thereof, as described herein, can at least partially fill the pores of the substrate. The coated articles generally can use any porous substrate, cellulosic or non-cellulosic, for example porous metal substrates, porous plastic (e.g., polymeric foam) substrates, and porous cellulosic substrates. A cellulosic substrate generally includes at least one of cellulose and hemicellulose, and it can further include lignin (e.g., as a lignocellulosic substrate).

In general, when the substrate is a cellulosic substrate, the cellulosic substrate is not particularly limited, and can be formed from any cellulosic material desired for protection with a synthetic wax coating. For example, the substrate can be a molded fiber containers, paper, paperboard, wood, or fabric (or textile). Examples of paper substrates can include, but are not limited to, generally thinner, flexible papers, for example useful as wrapping materials, as well as generally thicker, rigid papers or cardboard (e.g., corrugated paper cardboard, paperboards), for example useful as box, container, plate, cup, or other storage or food-service items. Suitable wood materials can be any type of wood commonly used in home, office, and outdoor settings. Suitable fabric or textile materials can include any cellulosic materials commonly used in garments or otherwise, such as cotton, jute, flax, hemp, etc.

In embodiments, the porous substrate includes a porous cellulosic substrate. In embodiments, the cellulosic substrate includes paper, corrugated board, cardboard, wood, fabric, and any combination thereof. The cellulosic substrate can be selected from the group of paper (bleached, unbleached, coated (pores still remain) and uncoated, supercallendered), corrugated board, cardboard, wood, and fabric (or textile). In some embodiments, the cellulosic substrate is in the form of a packaging box (e.g., corrugated boxes, cardboard boxes, cartons).

In embodiments (not shown), the substrate 310 has opposing first and second surfaces, and both surfaces of the substrate are coated with a synthetic wax coating 320 as described herein. The coatings 320 on opposing surfaces can be the same as or different from each other.

In other embodiments (not shown), two opposing substrates 310 can be adhered to or otherwise joined together by an intervening synthetic wax coating 320, for example where the synthetic wax serves as an adhesive. The substrates 310 can be the same as or different from each other.

The coating 320 can further include an additive (e.g., a filler). Examples of suitable additives include, but are not limited to, nanoclays, graphene oxide, graphene, silicon dioxide (silica), aluminum oxide, cellulose nanocrystals, carbon nanotubes, titanium dioxide (titania), diatomaceous earth, biocides, pigments, dyes, and thermoplastics. The additives can be included in any one layer or all layers of the synthetic wax coating as applied to the (porous and/or cellulosic) substrate. For example, the additives can be included in a solution or mixture containing the synthetic wax before it is applied to the substrate. Advantageously, the additives (e.g., fillers) can aid in sealing the substrate pores. Also, fillers can bring color to the substrate (e.g., paper), for example using titanium dioxide filler particles as a whitening agent. Biocidal properties can also be incorporated via nanofiber fillers. Other functions of the fillers (such as antioxidants, vitamin E, anti-fungals) include increasing the shelf-life and nutritional value of the product inside the coated paper. In addition, the first and/or second layers can be loaded with active components that kill certain microorganisms (e.g., bacteria, fungi or other microorganism) such as cimmaldehyde, carvacrol, sorbic acid, and nisin. Furthermore, cellulose nanocrystals, graphene, nanoclay, etc. as fillers can increase the gas and water vapor barrier properties. In embodiments, the coating includes one or more additives selected from the group consisting of nanoclay, graphene oxide, graphene, silicon dioxide (silica), aluminum oxide, cellulose nanocrystals, carbon nanotubes, titanium dioxide (titania), diatomaceous earth, biocides, pigments, dyes, thermoplastics, and combinations thereof. The various fillers and additives can be present in any suitable amount, for example at least 0.001, 0.01, 0.1, 0.2, 0.3, 0.5, 1, 2, 3, or 5 wt. % and/or up to 0.1, 0.2, 0.3, 0.5, 1, 2, 3, 5, 10, 15, or 20 wt. % relative to the coating. The foregoing amounts and ranges can independently apply to all fillers and additives collectively or to individual fillers or additives.

In embodiments, the coated article 300 can have a kit rating in a range of 4 to 12; and/or the coated article has a. For example, the kit rating can be at least 4, 5, 6, 7, 8, 9, 10, or 11 and/or up to 8, 9, 10, 11, or 12. Suitable methods for determining the kit rating include TAPPI methods T599 μm-96 and UM 557.

In embodiments, the coated article 300 can have a cobb (or cobb1800) rating of 20 g/m² or less. For example, the cobb rating (or cobb1800 rating) can be at least 0.1, 0.2, 0.5, 1, 2, 3, 6, 8, 10, 12, or 15 g/m² and/or up to 3, 5, 7, 10, 15, or 20 g/m². A suitable methods for determining the cobb rating includes TAPPI method T441 om-09.

In embodiments, the coated article 300 can have a relative permeability for water vapor of 0.5 or less, relative to a corresponding (porous) substrate without the coating thereon. For example, the coated article can have a relative permeability for water vapor of at least 0.00001, 0.0001, 0.001, 0.01, 0.1, 0.2, or 0.3 and/or up to 0.3, 0.4, or 0.5, such as 0.00001, 0.00005, 0.0001, 0.0005, 0.001, 0.005, 0.01, 0.05, 0.1, 0.2, 0.3, 0.4, or 0.5, relative to a corresponding (porous) substrate without the coating thereon (e.g., determined as a ratio of two water vapor transmission rate (WVTR) values). That is, the coated article can have a relative permeability for water vapor of 0.5 or less based on absolute water vapor transmission rates for the coated article and uncoated (porous) substrate. Alternatively or additionally, the coated article can have a relative permeability for non-water gas of at least 0.00001, 0.0001, 0.001, 0.01, 0.1, 0.2, or 0.3 and/or up to 0.3, 0.4, or 0.5, such as 0.00001, 0.00005, 0.0001, 0.0005, 0.001, 0.005, 0.01, 0.05, 0.1, 0.2, 0.3, 0.4, or 0.5, relative to a corresponding (porous) substrate without the coating thereon. This relative permeability for non-water gas can be applicable for one or more gases such as oxygen, nitrogen, carbon dioxide, and other common components of air. Alternatively or additionally, the coated article 300 can have an absolute permeability for water vapor of up to 100 g/m²/day, for example at least 0.1, 1, 2, 5, 7, 10, or 15 g/m²/day and/or up to 10, 15, 20, 25, 30, 40, 50, 60, 80, or 100 g/m²/day.

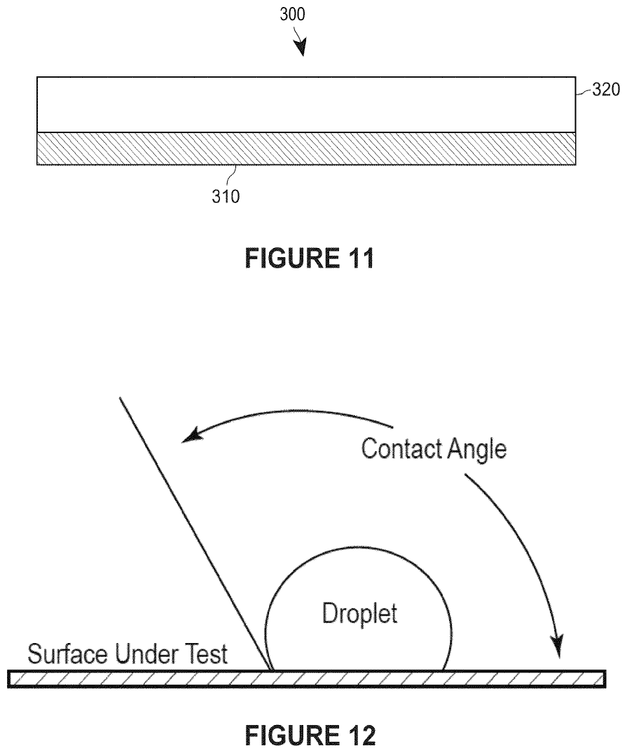

The water- and oil-resistance properties of the coated article 300 or corresponding coating 320 can be characterized in terms of one or more contact angles for water and/or oil droplets (e.g., vegetable oil such as castor oil) on the coating 320. FIG. 12 illustrates a contact angle for test droplet (e.g., water or oil droplet) on a generic substrate, which could be the coating 320 on an underlying substrate 310.

In embodiments, the article or coating has a water contact angle in a range from 80° or 90° to 120°, for example at least 80°, 85°, 90°, 95°, 100°, or 105° and/or up to 110°, 115°, or 120°, such as 90°, 95°, 100°, 105°, 110°, 115°, or 120°. The water contact angle can apply, for example, to a measurement after an interval of 30 sec or 5 min after application of a test droplet on the coating surface. In some cases, the water contact angle can be up to about 125° for non-smooth or rough surfaces.

In embodiments, the article or coating is resistant to the spreading of oil on its surface. In embodiments, the article or coating has an oil contact angle in a range from 1° to 65° or 10° to 75°, for example at least 1°, 10°, 20°, 30°, 40°, or 50° and/or up to 40°, 50°, 60°, 65°, 70°, or 75°. The oil contact angle can apply, for example, to a measurement after an interval of 30 sec or 5 min after application of a test droplet on the coating surface.

The contact angles for the article or coating can be higher when additives or nanofillers (e.g., clay, silica, etc.) are included in the composition as compared to a corresponding composition without any nanofillers. For example, in the case of articles or coatings further including one or more additives nanofillers (e.g., nanoclay, graphene oxide, graphene, silicon dioxide (silica), aluminum oxide, cellulose nanocrystals, carbon nanotubes, titanium dioxide), the contact angles suitably can range from 100° to 150° for water (e.g., at least 100°, 110°, 120°, 130° or 140° and/or up to 150°, 140°, 130°, 120°, or) 110°, and from 20° to 120° for oil (e.g., at least 20°, 30°, 40°, 50°, 60°, 70°, and/or up to 80°, 90°, 100°, 110°, or) 120°.

### Test Methods

Water Resistance: The water resistance of a wax can be measured as a cobb1800 value that represents grams of water per square meter that a wax absorbs in 1800 seconds when brought in contact with water. Cobb 1800 values were determined via a TAPPI standard T441 om-09 protocol, where a Cobb sizing tester (Büchel BV Inc. Utrecht, Netherlands) was used to allow DI water (100 mL) to come into contact with a 100-cm² or 133-cm² specimen for 1800 seconds (30 minutes). The weight of the water absorbed by the wax was calculated by the difference in the weight of each specimen before and after the test. Cobb 1800 values are expressed herein in grams per square meter (g/m²) unless otherwise indicated.

Oil/Grease Resistance (Kit Rating): Oil/grease resistance tests were performed in accordance with the T 559 μm-96 standard method or the TAPPI UM 557 standard method. Oil/grease resistance is represented by a kit rating value, where 12/12 denotes the maximum grease resistance, and 0/12 corresponds to no grease resistance. According to the methods, a series of numbered solutions (1-12) with various surface tensions and viscosities (aggressiveness) were prepared by mixing specific proportions of castor oil, n-heptane, and toluene. Higher numbered solutions are more aggressive with lower surface energies (i.e., solution #1 is the least aggressive oil while #12 is the most aggressive oil). A test specimen was placed on a black bench, and various test solutions were gently allowed to drop onto the surface of the specimen from a height of 0.5 inches and quickly removed with a clean tissue after 15 s. The tested area was examined immediately and a specimen with darkened spots was considered to have failed the test. The number of the most aggressive solution that remained on the surface of a specimen without causing any failure was reported as the “kit rating.” A higher kit rating indicates stronger grease resistance.

Preparation of coated paper: 1.0 g of the polymeric wax was introduced into a 20 ml vial followed by the addition of 2 ml chloroform. The mixture was kept on stirring to obtain a clear solution. The obtained clear mixture was cast onto kraft paper (pre-coated with 5% starch) using a silicon spatula to get a smooth and uniform coating. The coated paper was subsequently dried in oven at 60° C. for 30 minutes, followed by drying at room temperature for 24 hrs. The coatings generally had a coating loading of about 40-60 g/m² or about 50-55 g/m².

Nuclear Magnetic Resonance (NMR) Analysis: ¹H-NMR spectra for all samples were recorded using 500 MHz NMR spectrometer. Deuterated chloroform (CDCl3) and water (D2O) were used as solvents for all samples. Chemical shift values for all the spectrums were recorded in ppm.

Fourier-transform Infrared (FTIR) analysis: FTIR analysis for all the samples was done using FT/IR-6600 spectrometer designed by JASCO (Easton, Maryland, USA). The FTIR spectra were recorded using 32 scans in the range of 500-4000 cm⁻¹ at room temperature using resolution of 4 cm⁻¹.

Differential scanning calorimetry analysis (DSC): Differential scanning calorimetry analysis was done using DSC Q100 model instrument 10-15 mg of the sample was taken for each polymeric wax under the nitrogen flow of 100 ml/min. The three cycles method was used, and temperature was kept in between the range of 0° C. to 200° C. at a rate of 10° C./min for all samples.

Thermogravimetric analysis (TGA): Thermogravimetric analysis of all the samples were recorded using thermogravimetric analyzer (Q 50). 8-12 mg of the sample was taken in a platinum pan using nitrogen flow of 40 ml/min. The temperature was kept from 25° C. to 600° C. using a heating rate of 10° C./min.

Water vapor transmission rates analysis (WVTR): A PERMATRAN-W system (Model 3/34, Mocon Inc., MN, USA) was used to determine water vapor transmission rates (WVTR) at 23° C. and at 50% RH as well as at 90% RH and 38° C. Water vapor permeation was calculated by multiplying thickness of paper samples with water vapor transmission values.

Contact angle measurements: An approximately 10 μL droplet of deionized water or castor oil was introduced on the tested paper samples. A 590-U1 AST VCA 2500XE Video Contact Surface Inspection Goniometer Fuji 611847 (AST Products, Inc. MA, USA) instrument was used for contact angle analysis. The images were recorded after placing the droplet and at different time intervals i.e., 30 sec and 5 min, generally at about room temperature (e.g., 20-30° C. or about 25° C.). The tested surface was also examined to trace the appearance of any dark stains once the test was completed. The contact angles were taken in triplicates and results were reported as mean of left and right angles.

Thermal sealing: The ASTM F88-21 standard protocol was adopted for recording thermal seal strength. The sealed samples were prepared using a bar thermal sealer (SENCORP, MA, USA). The 4-inch-long (about 10 cm) and 1-inch-wide (about 2.5 cm) sample stripes were taken for preparing sealed samples at 121° C. (250° F.) keeping sealing time at 5 second and sealing width of 0.4 inches (about 1 cm). The sealed samples were stored at 50% relative humidity and room temperature for 24 hours prior testing their seal strength. The maximum seal strength was recorded by measuring the force (N) required to break the seal and tensile strength (MPa) at maximum load and break point using 5565 Universal Instron Testing Machine (Instron, MA, USA). The grip separation rate was maintained to 10 inches/minute and grip separation of one inch. The samples were produced in triplicate.

Chemical degradation: 250 mg of each polymeric wax was taken in a 10 ml vial followed by the addition of 2 ml of 5 weight % of Na₂CO₃ solution. It was noted when the polymer's particles started to scatter in an alkaline medium. For each sample, the moment when it entirely disperses was the time for its degradation.

Recyclability: 2 g of a selected wax-coated paper was cut into small pieces and soaked into 50 mL of 3% Na₂CO₃ solution and was kept at 75° C. for 10 minutes. The soaked coated paper was then kept at room temperature for 1 hr. The liquid was decanted followed by washing paper with deionized water three times to remove any suspended coating material left behind. The recycled paper was then dried in a vacuum oven for 24 hrs. at 70° C.

Scanning electron microscopy (SEM): The SEM analysis was performed using JEOL SEM System (6610), to explore the surface morphology of paper samples. Prior to SEM analysis, each sample was loaded with a thin layer of gold (15 nm) using sputtering technique.

Compression Molding: 5 g of polymeric wax sample was spread on 5% starch-coated paper sample and was pressed using PHI Manual Hydraulic Compression Press (California, USA) at 70° C. for 4 minutes to obtain a coated paper sample.

Gel permeation chromatography (GPC) analysis: Gel permeation chromatography (GPC) analysis was performed using refractive index detector (Waters 2414) and Isocratic HPLC pump (Waters 1515) connected to plus Autosampler (Waters 717). The polymeric wax sample (P-W-LA3) was dissolved in tetrahydrofuran (2 mg/mL). Approximately 100 μL of the solutions (filtered) were injected and adjusted the run time=50 min and flow rate=1.0 mL/min. The instrument was calibrated using PS (polystyrene) standards. A Waters BREEZE Software was used for the molecular weight distribution (MWD) curves.

### EXAMPLES

The following examples illustrate the disclosed synthetic waxes and methods for synthesizing same, but are not intended to limit the scope of any claims thereto. Equivalents (eq.) are listed as molar equivalents unless indicated otherwise.

### Example 1: Synthesis of Biodegradable Synthetic Waxes

This example illustrates the synthesis of various wax materials according to the disclosure as well as comparative wax materials. Various wax materials were tested for their water resistance (cobb1800 value) and oil/grease resistance (kit rating) when applied as a coating on a paper substrate. In some cases, a blend with 90 wt. % synthetic wax and 10 wt. % carnauba wax was tested and was shown to have excellent water and oil resistance properties. In some cases, fillers such as calcium carbonate were added to the synthetic wax coating, also improving the water and oil resistance properties.

The prepared waxes according to the disclosure generally had the structure (A-a-B-b-C), where A is a long chain alkyl group, B is an oligo/polyester (in particular with degradable lactic and/or glycolic acid units), and C is long chain alkyl group, a short chain alkyl group, or just a hydroxyl (OH) group. The units a and b are optional; when present, a and b are linking groups that connect A-B and B-C, respectively. The a and b linkers are often ester linkages. In certain cases, B includes a rapidly degradable group such as a glycolic ester relevant for recycling. Blends of the wax A-a-B-b-C with 10% or less fillers (e.g., such as natural wax, calcium carbonate, other polymers) can improve performance such as thermal resistance and water resistance. The wax A-a-B-b-C can be applied on paper as a solution (e.g., water borne or solvent borne for solution casting) on in melt form. The paper can be optionally coated with starch or another hydrophilic polymer (i.e., onto which the synthetic wax is coated, such as in a paper-starch-synthetic wax arrangement). The wax A-a-B-b-C can be 100% biodegradable. The wax A-a-B-b-C also can be degradable in numerous solutions such as sodium hydroxide, sodium carbonate, sodium bicarbonate, sodium silicate, polyethylene imine (e.g., to facilitate recycling and/or re-use of the wax and/or the substrate to which it is coated, such as paper).

Table 1 below provides illustrative chemical structure of tested materials along with their cobb1800 value and kit rating. The samples were generally dried in oven at 60° C., followed by drying at room temperature (about 25° C.) for 2 hours. Certain samples (denoted with “*”) were kept at room temperature (about 25° C.) for 24 hours prior to kit and cobb testing.

TABLE 1
Wax Chemical Structure and Resistance Values for Wax-Coated Paper

| | | Cobb | |
|---|---|---|---|
| | | 1800 | |
| Ex. | Structure | (g/m²) | Kit |
| | Starch-coated Kraft paper (control) | 87 | 0 |
| PH71 | 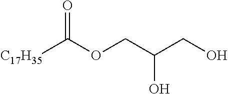 | 136 | 5 |
| PH73 | 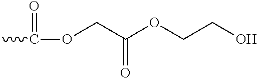 | 6.2 | 7 |
| PH74 | 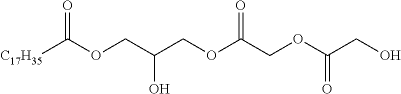 | 152.6 | 5 |
| PH75 | 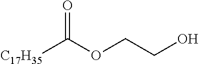 | 88.8 | 3 |
| PH76 | 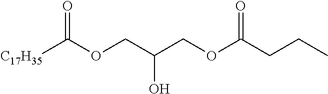 | 46.2 | 4 |
| PH77 | 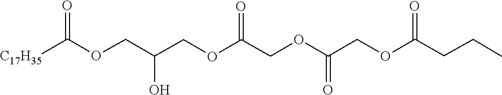 | 36.5 | 12 |
| PH78 | 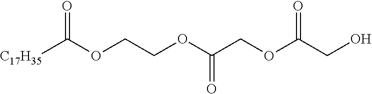 | Swelled in 7 mins | 1 |
| PH79 | 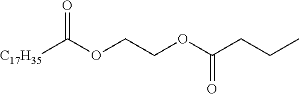 | 42.6 | 10 |
| PH80 | 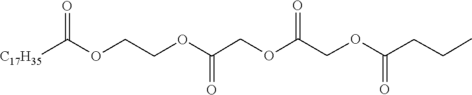 | 39.3 | 10 |
| M3 | 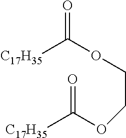 | Swelled in 8 mins | 10 |
| M5 | 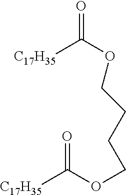 | 69 | 5 |
| AJ1 | 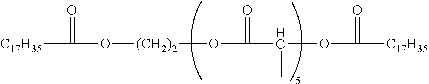 Stearate-EG-OLA₅-Stearate | 29, 29.3* | 12, 12* |
| AJ2 | 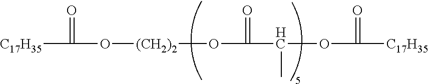 Stearate-EG-OLA₅-Stearate + Carnauba wax (10 wt %) | 18, 25.9* | 12, 12* |
| AJ3 | 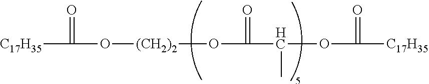 Stearate-EG-OLA₅-Stearate | 21, 8.8* | 12, 12* |
| AJ4 | 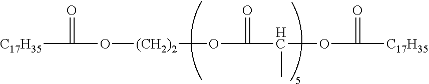 Stearate-EG-OLA5-Stearate + Carnauba wax (10 wt %) | 4.7, 4.4* | 12, 12* |
| AJ5 | 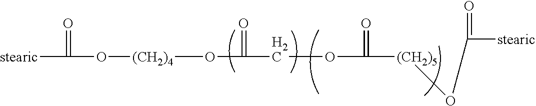 Stearic-EG-OG1LACTONE5-Stearic | 74, 16* | 5, 12* |
| AJ6 | 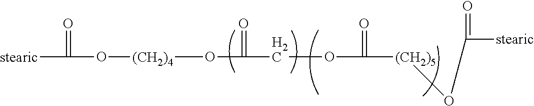 Stearic-EG-OG1LACTONE5-Stearic + Carnauba wax (10 wt %) | 75, 13* | 7, 12* |
| Wax- 12.1 | 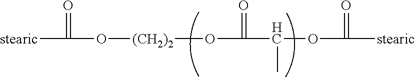 Stearate-EG-OLA₉-Stearate | Not tested | Not tested |
| Wax- 12.3 | 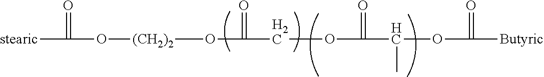 Stearate-EG-OG₁LA₅-Butryic | Not tested | Not tested |
| Wax- 12.3 | 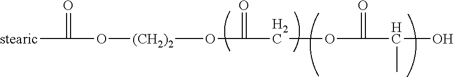 Stearate-EG-OG₁LA₅-OH | Not tested | Not tested |

Tables 2 and 3 below provide cobb1800 value and kit rating results for additionally tested wax materials. The wax-coated samples were generally formed by applying synthetic wax solution in chloroform (1:2 w/w wax:chloroform) onto 5 wt % starch coated kraft paper, and then dried in oven at 60° C., followed by drying at room temperature (about 25° C.) for 24 hours. In Tables 2 and 3, cobb1800 and kit results in parentheses reflect a blend of about 90.9 wt. % of the indicated synthetic wax and about 9.1 wt. % carnauba wax.

TABLE 2
Resistance Values for Wax-Coated Paper

| | | Cobb | |
|---|---|---|---|
| | | 1800 | |
| Ex. | Structure | (g/m²) | Kit |
| Aj11 | 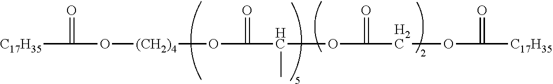 | 14.5 (4.4) | 12 (12) |
| Aj13 | 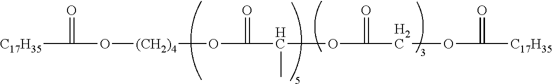 | 14.7 (5.6) | 12 (12) |
| Aj15 | 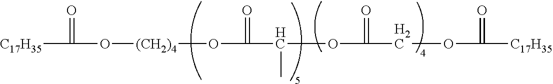 | 11.8 (5.4) | 12 (12) |
| Aj17 | 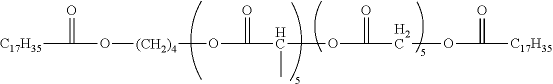 | 19 (3.27) | 11 (12) |
| Aj19 | 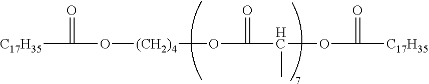 | 6 (2.9) | 12 (12) |
| Aj21 | 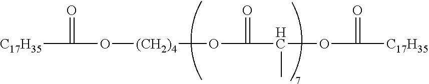 90.9 wt. % Aj21 wax and 9.1 wt. % calcium carbonate | 2 | 12 |

TABLE 3
Resistance Values for Wax-Coated Paper

| | | Cobb | |
|---|---|---|---|
| | | 1800 | |
| Ex. | Structure | (g/m²) | Kit |
| M12 | 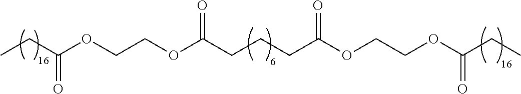 | 65.9 (63) | 10 (9) |
| M13 | 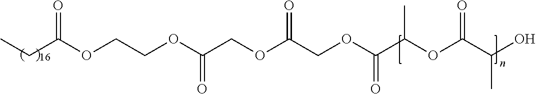 | 5.1 (3.5) | 12 (12) |
| M14 | Stearic acid (1 mole equiv.) + Adipic acid (1 mole equiv.) + Ethylene glycol (1.5 mole equiv.) 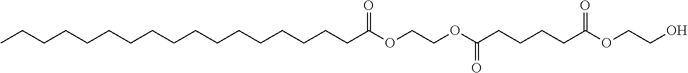 [SMILES: CCCCCCCCCCCCCCCCCC(=O)OCCOC(=O)CCCCC(=O)OCCO] | 64.8 (60.8) | 5 (8) |
| M15 | Stearic acid (1 mole equiv.) + Adipic acid (2 mole equiv.) + Ethylene glycol (2.5 mole equiv.) 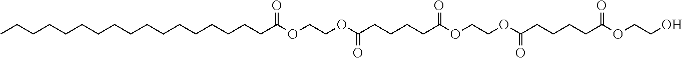 [SMILES: CCCCCCCCCCCCCCCCCC(=O)OCCOC(=O)CCCCC(=O)OCCOC(=O)CCCCC(=O)OCCO] | 124.2 (111.5) | 0 (0) |
| PH8 | 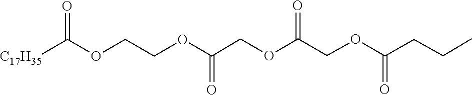 | 36.4 (10.1) | 12 (12) |

### Example 2: Synthesis of Stearate-EG-Glycolide-Lactide-DSA Synthetic Wax

2-Hydroxyethylstearate was synthesized by mixing stearic acid with excess ethylene glycol and 1.0 wt % tin 2-ethylhexanoate at 150° C. for 5-6 hr, cooling the reaction mixture to about 25° C., and rinsing the resulting white solid with excess water to remove unreacted ethylene glycol. The solid was dissolved in chloroform and dried with MgSO₄ prior to use.

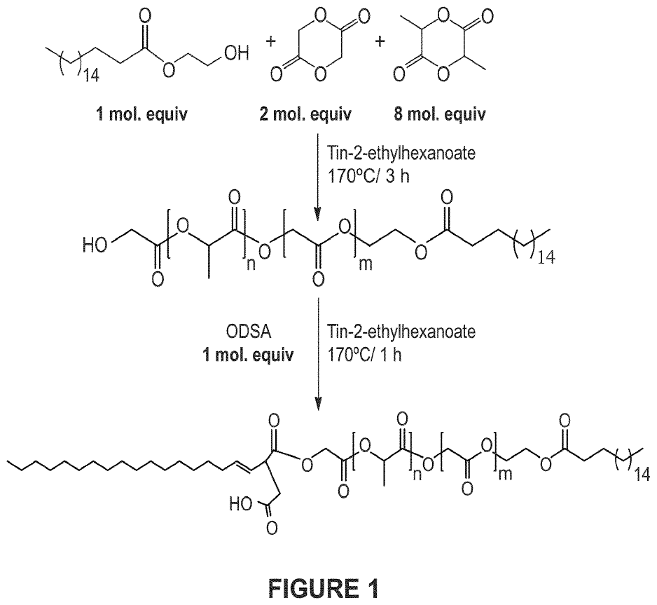

A mixture of 2-hydroxyethyl stearate (1 eq.), glycolide (2 eq.), lactide (8 eq.), and tin 2-ethylhexanoate (0.5 wt. %) was heated at 170° C. for 3 hr in a sealed pressure tube, then cooled to room temperature (about 25° C.). To the cooled mixture of the first step was added a mixture of (2-dodecen-1-yl) succinic anhydride (DSA) (1 eq., based on the amount of mixture resulting from the first step) and tin 2-ethylhexanoate (0.5 wt. %). The resulting mixture was heated at 170° C. for 1 h, yielding stearate-EG-glycolide-lactide-DSA synthetic wax. A schematic for an analog of this synthesis is shown in FIG. 1 using octadecenylsuccinic anhydride (ODSA) in place of DSA.

### Example 3: Synthesis of ODSA-Lactide-EG-Lactide-ODSA Synthetic Wax

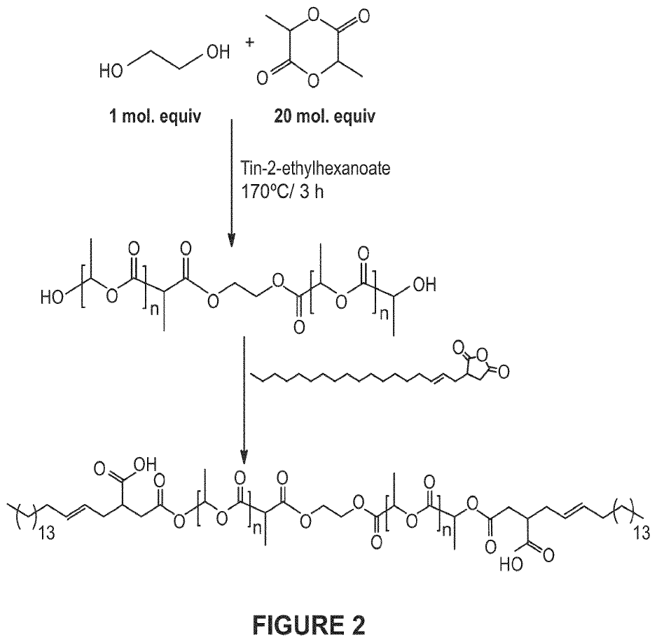

As a first step, a mixture of lactide (20 eq.), ethylene glycol (1 eq.) and tin 2-ethylhexanoate (0.5 wt. %) was heated at 170° C. for 3 hr in a sealed pressure tube, then cooled to ~25° C. To the cooled mixture of the first step was added a mixture of octadecenylsuccinic anhydride (ODSA) (2 eq., based on the amount of mixture of the first step) and tin 2-ethylhexanoate (0.5 wt. %). The resulting mixture was heated at 170° C. for 1 hr, yielding ODSA-lactide-EG-lactide-ODSA synthetic wax. A schematic of the synthesis is shown in FIG. 2.

### Example 4: Synthesis of DSA-Lactide-EG-Lactide-DSA Synthetic Wax

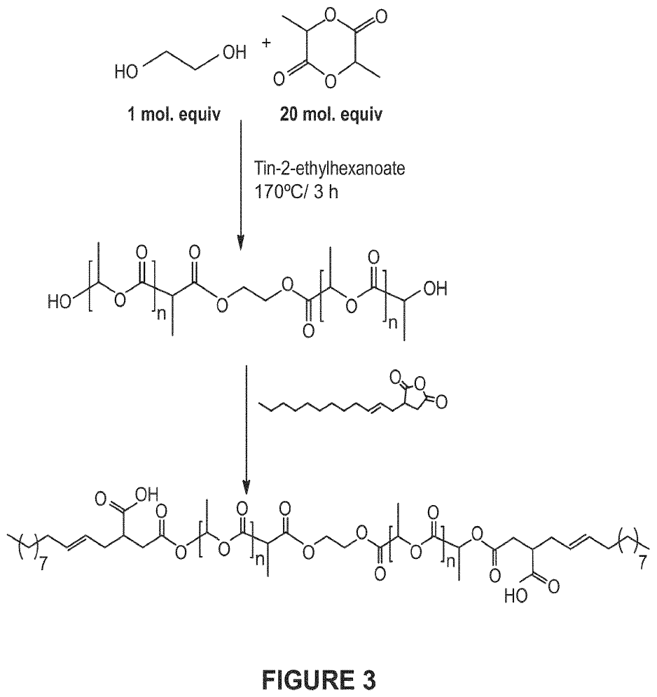

As a first step, a mixture of lactide (20 eq.), ethylene glycol (1 eq.) and tin 2-ethylhexanoate (0.5 wt. %) was heated at 170° C. for 3 hr in a sealed pressure tube and cooled to ~25° C. To the cooled mixture of the first step was added a mixture of (2-dodecen-1-yl) succinic anhydride (2 eq., based on the amount of mixture of the first step) and tin 2-ethylhexanoate (0.5 wt. %). The resulting mixture was heated at 170° C. for 1 hr, yielding DSA-lactide-EG-lactide-DSA synthetic wax. A schematic of the synthesis is shown in FIG. 3.

The waxes of Examples 2-4 were tested for their cobb1800 value and kit rating as a paper coating, the results are shown in Table 4. The waxes of Examples 2-4 were also tested for the extent of emulsification achieved when the synthetic waxes were contacted with a sodium carbonate solution (250 mg wax sample in 2 ml of 5 wt. % of Na₂CO₃ aqueous solution). A 100% emulsification reflects the ease with which the waxes according to the disclosure can be removed from an existing substrate for recycling.

TABLE 4
Resistance and Emulsification Values and for Wax-Coated Paper

| | Cobb1800 | | |
|---|---|---|---|
| Example | (g/m²) | Kit | Emulsification % |
| 2 | 13.1 | 12 | 100% in Na₂CO₃ after 10 min at 60° C. |
| 3 | 8.31 | 12 | 60% in Na₂CO₃ after 15 min at 60° C. |
| 4 | 3 | 12 | 100% in Na₂CO₃ after 15 min at 60° C. |

### Example 5: Synthesis of High-Melting Polymeric Synthetic Wax

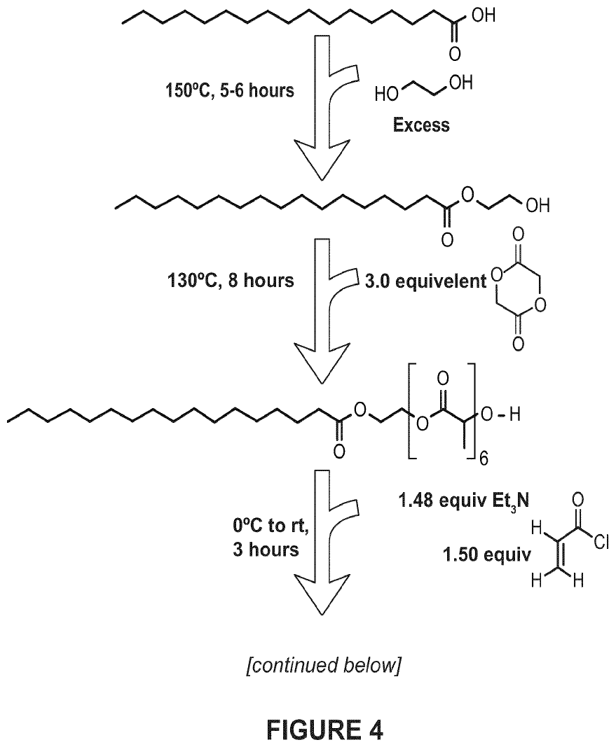

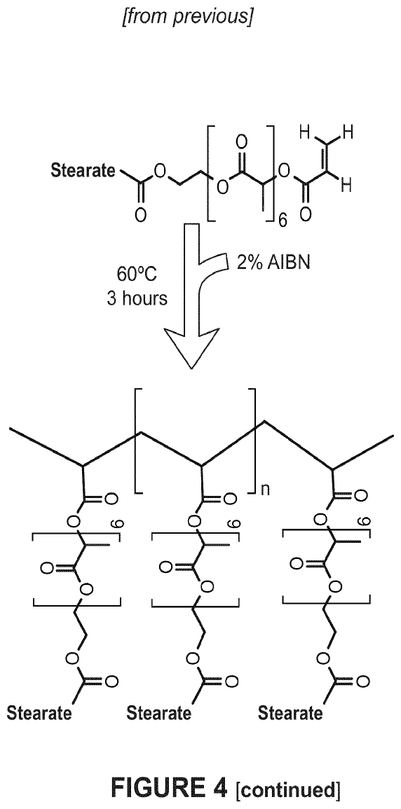

Synthetic waxes were formed by reacting 2-hydroxyethylstearate (stearate-EG) with one or both of lactide and glycolide, the product of which was then acrylated with acryloyl chloride to form a monomeric synthetic wax. The product was then subjected to a vinyl polymerization via the unsaturated acrylate groups to form a corresponding high-melting polymeric synthetic wax. Table 5 shows the relative proportions of stearate-EG, lactide, and glycolide in different synthesized samples. An illustrative reaction scheme is shown in FIG. 4, and specific reaction steps are described in more detail below.

TABLE 5
Proportions of Stearate-EG, Lactide, and Glycolide in High-Melting Waxes

| Sample | Name | Stearate-EG | Lactide | Glycolide |
|---|---|---|---|---|
| 1 | P-W-LA0 | 1.0 eq. | — | — |
| 2 | P-W-LA3 | 1.0 eq. | 3.0 eq. | — |
| 3 | P-W-LA4 | 1.0 eq. | 4.0 eq. | — |
| 4 | P-W-LA4/1 | 1.0 eq. | 4.0 eq. | 1.0 eq. |
| 5 | P-W-LA5 | 1.0 eq. | 5.0 eq. | — |
| 6 | P-W-LA7 | 1.0 eq. | 7.0 eq. | — |
| 7 | P-W-LA7/1 | 1.0 eq. | 7.0 eq. | 1.0 eq. |
| 8 | P-W-LA10 | 1.0 eq. | 10.0 eq. | — |

Preparation of stearate-EG (FIG. 4, first reaction): A 250 mL flask was charged with 50 grams of stearic acid, ethylene glycol (100 mL) and 1% wt. Tn (II) ethyl hexanoate (500 mg). The flask was connected to the deanstark apparatus and set the reaction temperature to 150° C. The reaction mixture was stirred for 5 to 6 hours after which the mixture was transferred to a beaker. Once the reaction mixture cools down to room temperature, the white solid on the top was washed with hot water (3×600 mL) to remove ethylene glycol content. Each time the water was removed when it was cooled to rt. The solid white precipitate was next dissolved in chloroform and dried with MgSO₄ before further use.

Preparation of stearate-EG-PLA (FIG. 4, second reaction): A 250 mL flask was charged with 20.00 grams of stearate-EG (57.00 mmol) followed by vacuum drying at 80° C. for 2 hours. Next, lactide (25.00 grams, 172 mmol) was added to the flask and further dried at 80° C. for 1 hour. Subsequently, the vacuum was removed by nitrogen flow and the reaction mixture was added 1 wt. % Zn(II) ethyl hexanoate (200 mg). The temperature of the reaction was increased to 125-130° C. and stirred for 8 hours. An off-white gel type of substrate was recovered from the flask.

Preparation of stearate-EG-PLA-acrylate (FIG. 4, third reaction): A 250 mL flask was charged with 45.00 grams of stearate-EG-PLA (about 57.0 mmol) pre-dissolved in THF or CHCl₃ (100 mL). The reaction mixture was cooled to 0° C. by placing it in ice bath. Next, triethylamine (1.48 equiv, 11.75 mL) was added, followed by dropwise addition of acryloyl chloride (1.50 equiv, 6.91 mL). Subsequently, the reaction mixture was brought to room temperature and stirred for 3 hours. Once the reaction was completed, the reaction mixture was poured into hot distilled water (600 mL). The gel precipitate was next dissolved in chloroform (200 mL) and extracted with water (3×600 mL). The chloroform extract was dried with MgSO₄ followed by solvent evaporation.

Preparation of polymeric wax (FIG. 4, fourth reaction): A 25 mL flask was charged with 4 grams of stearate-EG-PLA-acrylate (about 5.0 mmol) and AIBN (2 wt. %, 80 mg). The flask was vacuumed for several minutes followed by nitrogen flow. Next, 2 mL of THF (degassed) was also added to the reaction mixture under nitrogen flow. The reaction mixture was heated up to 60° C. and stirred for 3 hours.

The polymeric waxes were variously tested for melting properties via differential scanning calorimetry (DSC). The polymeric waxes were also coated on a paper substrate and tested for their resistance properties, including water vapor transmission rate (WVTR), cobb1800 water resistance, kit rating oil/grease resistance, water contact angle (“WCA”; 30 sec and 5 min contact time), and oil contact angle (“OCA”; 30 sec and 5 min contact time). The resistance properties were compared with uncoated kraft paper (“KR”) and kraft paper with 5% starch coating (“KRS”) (but no wax coatings) as control samples. The results are shown in Table 6.

TABLE 6
Melting and Resistance Values and for Synthetic Waxes

| | | Cobb1800 | | WVTR | WCA | WCA | OCA | OCA |
|---|---|---|---|---|---|---|---|---|
| Sample | DSC mp (° C.) | (g/m²) | Kit | (g/m²/day) | (30 s) | (5 min) | (30 s) | (5 min) |
| KR | n/a | 164 | 0 | 817 | 39° | 0° | 35° | 27° |
| KRS | n/a | 86.5 | 9 | 400 | 58° | 44° | 33° | 19° |
| P-W-LA0 | ~60 | 23.7 | 12 | — | — | — | — | — |
| P-W-LA3 | ~67 | 4.10 | 12 | 28 | 86° | 76° | 56° | 50° |
| P-W-LA4 | — | 4.55 | 12 | 34 | 110° | 90° | 62° | 59° |
| P-W-LA4/1 | — | 8.35 | 12 | 45 | 98° | 88° | 67° | 63° |
| P-W-LA5 | ~107 | 15.1 | 12 | — | — | — | — | — |
| P-W-LA7 | ~117 | 28.9 | 12 | — | — | — | — | — |
| P-W-LA7/1 | ~116 | 11.1 | 12 | — | — | — | — | — |
| P-W-LA10 | ~123 | — | — | — | — | — | — | — |

Chemical Degradation. A chemical degradation test was performed for wax samples by using sodium carbonate as described above. Very similar tests were also performed for paraffin and carnauba wax, to see the degradation behavior for comparison. 250 mg of the waxes (P-W-LA3, paraffin, and carnauba) were taken in 2 mL of solution of 5% sodium carbonate. The polymeric wax with three LA units (P-W-LA3) had degraded in 7 min at 75° C. Sodium lactate, a water-soluble compound, was identified as the primary product resulting from the degradation of the polymeric wax. Additionally, the degraded product was subjected to chloroform extraction, revealing stearic-EG-OH as the main component in this fraction. GPC analyses were also conducted on the wax sample (P-W-LA3) before and after degradation. It was noted that the polymeric retention time (RT), previously noted for polymeric wax at 22.98 min, disappeared after the degradation process, suggesting that the degradation was successful. Other polymeric wax samples also underwent degradation in 5% sodium carbonate at 75° C., but the time required for the complete degradation to occur was different for each polymeric wax: 12 min (P-W-LA4), 18 min (P-W-LA4/1), 45 min (P-W-LA5), 110 min (P-W-LA7), and 45 min (P-W-LA/1). Accordingly, the P-W-LAn waxes were found to be degradable and suitable for packaging applications. In contrast, no dispersion was observed for paraffin and carnauba wax in alkaline medium, which indicated their nondegradable nature in the given conditions.

Recyclability: The recyclability of wax-coated paper was tested in which 2.0 g of the selected coated paper was cut into small pieces and soaked into 50 mL of 3 wt % sodium carbonate solution and was kept at 70 to 75° C. for 10 min. The soaked coated paper was then kept at room temperature for 1 h. The liquid was decanted, followed by washing paper with deionized water three times to remove any suspended coating material that was left behind. It was found that almost all of the coating material layers became separated from the paper. The paper was then dried in a vacuum oven for 24 h at 70° C. FTIR analysis of the recycled paper confirmed the separation of the coated material from the paper. The recovered paper's FTIR spectrum closely matched the uncoated commercial Kraft paper.

The polymeric wax corresponding to P-W-LA3 was tested for its thermal sealing properties to join two substrates. When heated to about 120° C. (or about 250° F.) for about 3 seconds or longer, the polymeric wax formed an excellent, strong seal between the substrates.

### Example 6: Synthesis of High-Melting Carboxylic Synthetic Wax

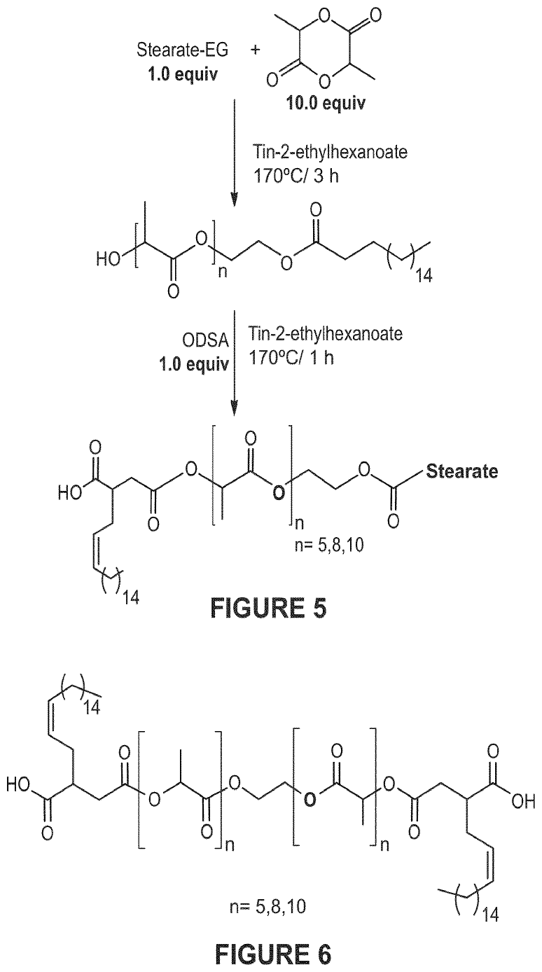

Synthetic waxes were formed by reacting stearate-EG with lactide, the product of which was then reacted with octadecenylsuccinic anhydride (ODSA). An illustrative reaction scheme is shown in FIG. 5: In step 1, a mixture of stearate-EG (1.0 equiv), lactide (10.0 equiv) and tin-2-ethyl hexanoate (0.5 wt. %) was heated at 170° C. for 3 h in a sealed pressure tube followed by cooling to room temperature. In step 2, ODSA (1.0 equiv) and tin (II)-ethyl-hexanoate (0.5 wt) were added were added to the product of step 1, and the reactant mixture was heated at 170° C. for 1 h. The resulting wax product contains one pendent carboxylic acid group (i.e., permitting complexation with Ca⁺² or other polyvalent metal) and one unsaturated C═C bond (i.e., permitting partial polymerization and/or crosslinking with peroxide.

Table 7 shows the relative proportions of stearate-EG, lactide, and ODSA in different synthesized samples. The samples were then tested for their physical melting point at 75° C. for (1) the product itself (i.e., monomeric synthetic wax), (2) a peroxide-polymerized analog of the product (i.e., polymeric synthetic wax), and (3) a calcium (Ca²⁺)-complex analog of the product (i.e., metal-complexed synthetic wax via carboxylic units of the monomeric synthetic wax). Table 6 indicates whether each of different wax sample melted or did not melt at 75° C.

TABLE 7
Proportions of Stearate-EG, Lactide, and ODSA in High-Melting Carboxylic Waxes

| | | | | | | | | Ca²⁺_{—} |
|---|---|---|---|---|---|---|---|---|
| | | Stearate- | | | | Monomer | Polymer | Complex |
| Sample | Name | EG | Lactide | | ODSA | Product | Product | Product |
| 1 | St-LA5- | 1.0 eq. | 5.0 | eq. | 1.0 | Melted | Melted | Not Melted |
| | ODSA | | | | eq. | | | |
| 2 | St-LA8- | 1.0 eq. | 8.0 | eq. | 1.0 | Not | Not | Not Melted |
| | ODSA | | | | eq. | Melted | Melted | |
| 3 | St-LA10- | 1.0 eq. | 10.0 | eq. | 1.0 | Not | Not | Not Melted |

| | | | |
|---|---|---|---|
| ODSA | eq. | Melted | Melted |

### Example 7: Synthesis of High-Melting Dicarboxylic Synthetic Wax

Synthetic waxes were formed by reacting ethylene glycol (EG) with lactide, the product of which was then reacted with octadecenylsuccinic anhydride (ODSA). In step 1, a mixture of lactide (20.0 equiv), ethylene glycol (1.0 equiv) and tin (II)-ethyl-hexanoate (0.5 wt) was heated at 170° C. for 3 h in a sealed pressure tube followed by cooling to room temperature. In step 2, ODSA (2.0 equiv) and tin (II)-ethyl-hexanoate (0.5 wt) were added were added to the product of step 1, and the reactant mixture was heated at 170° C. for 1 h. The resulting wax product contains two pendent carboxylic acid groups (i.e., permitting complexation with Ca⁺² or other polyvalent metal) and two unsaturated C═C bonds (i.e., permitting partial polymerization and/or crosslinking with peroxide. An illustrative structure is shown in FIG. 6.

Table 8 shows the relative proportions of EG, lactide, and ODSA in different synthesized samples. The samples were then tested for their physical melting point at 75° C. for (1) the product itself (i.e., monomeric synthetic wax), (2) a peroxide-polymerized analog of the product (i.e., polymeric synthetic wax), and (3) a calcium (Ca²⁺)-complex analog of the product (i.e., metal-complexed synthetic wax via carboxylic units of the monomeric synthetic wax). Table 8 indicates whether each of the different wax samples melted or did not melt at 75° C.

TABLE 8
Proportions of EG, Lactide, and ODSA in High-Melting Dicarboxylic Waxes

| | | | | | | | Ca²⁺- |
|---|---|---|---|---|---|---|---|
| | | | | | Monomer | Polymer | Complex |
| Sample | Name | EG | Lactide | ODSA | Product | Product | Product |
| 1 | ODSA-LA5-EG-LA5- | 1.0 | 10.0 | 2.0 | Melted | Melted | Not Melted |
| | ODSA | eq. | eq. | eq. | | | |
| 2 | ODSA-LA8-EG-LA8- | 1.0 | 16.0 | 2.0 | Melted | Melted | Not Melted |
| | ODSA | eq. | eq. | eq. | | | |
| 3 | ODSA-LA10-EG- | 1.0 | 20.0 | 2.0 | Melted | Not | Not Melted |
| | LA10-ODSA | eq. | eq. | eq | | Melted | |

### Example 8: Synthesis of Stearate-Ethylene Glycol Synthetic Wax

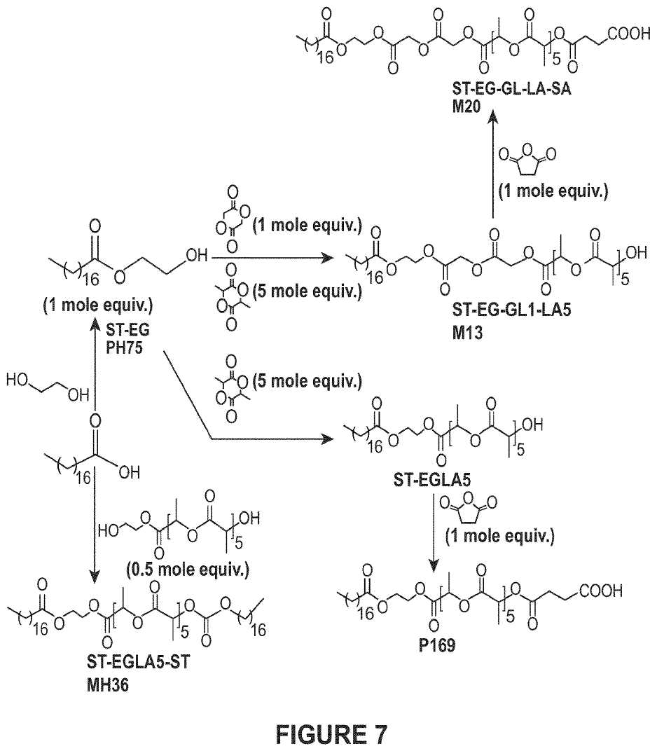

Synthetic waxes were formed by reacting stearate-EG with lactide and/or glycolide, along with one or more other co-reactants. All products were characterized by 1H NMR spectroscopy (500 MHz, Varian 7600-AS, USA). The samples were prepared by dissolving 2-3 mg of each sample in 0.7 mL of chloroform-d (CDCl₃). The general synthetic methods are illustrated in FIG. 7 and described in more detail below.

Synthesis 2-hydroxyethyl stearate (ST-EG) (PH75): A mixture of stearic acid (50 g, 1 mole equiv.), EG (218 g, 20 mole equiv.) and zinc acetate dihydrate (2 wt. %) was heated at 200° C. for 15 h. The reaction was cooled to r.t. (about 25° C.), water was added to remove the excess of ethylene glycol, and the product was extracted over chloroform with a yield of 98%. ST-EG ¹H-NMR (CDCl₃): δ (ppm)=0.87 (t, CH₃), 1.24-1.29 (b, 14 CH₂), 1.59-167 (m, CH₂ CH₂CO), 2.34 (t, CH₂CO), 3.83 (t, CH₂OH of EG), 4.19 (t, CH₂OCO of EG).

Synthesis of stearic acid-ethylene glycol-glycolide-lactide (ST-EG-GL1-LA5) (M13): A mixture of ST-EG (20 g, 1 mole equ.), GL (7.1 g, 1 mole equ.), LA (44 g, 5 mole equ.) and tin(II) 2-ethylhexanoate (0.5 wt. %) was heated at 170° C. for 3 h. The reaction was cooled to r.t. to give the product a yield of 99%. ST-EG-GL1-LA5 ¹H-NMR (CDCl₃): δ (ppm)=0.87 (t, CH₃), 1.24-1.30 (b, 14 CH₂), 1.48-1.68 (m, CH₂CH₂CO and CH₃ of LA), 2.31 (t, CH₂CO), 4.26-4.42 (m, 2CH₂O of EG), 4.59-4.89 (m, 2CH₂O of GL), 5.13-5.25 (m, CHO of LA).

Synthesis of stearic acid-ethylene glycol-glycolide-lactide-succinic anhydride (ST-EG-GL1-LA5-SA) (M20): A mixture of (ST-EG-GL1-LA5) (30 g, 1 mole equ.), succinic anhydride (2.57 g, 1 mole equ.) and tin(II) 2-ethylhexanoate (0.5 wt. %) was heated at 130° C. for 1 h. The reaction was cooled to r.t. to give the product a yield of 99%. ST-EG-GL1-LA5-SA ¹H-NMR (CDCl₃): δ (ppm)=0.87 (t, CH₃), 1.24-1.28 (b, 14 CH₂), 1.52-1.67 (m, CH₂CH₂CO and CH₃ of LA), 2.31 (t, CH₂CO), 2.70, 2.99 (b, 2 CH₂ of SA), 4.26-4.38 (m, 2CH₂O of EG), 4.59-4.89 (m, 2CH₂O of GL), 5.13-5.21 (m, CHO of LA).

Synthesis of stearic acid-ethylene glycol-glycolide-lactide (ST-EG-LA5): A mixture of ST-EG (20 g, 1 mole equ.), LA (44 g, 5 mole equ.) and tin(II) 2-ethylhexanoate (0.5 wt. %) was heated at 170° C. for 3 h. The reaction was cooled to r.t. to give the product a yield of 98%. ST-EG-LA5 ¹H-NMR (CDCl₃): δ (ppm)=0.87 (t, CH₃), 1.23-1.27 (b, 14 CH₂), 1.47-1.60 (m, CH₂CH₂CO and CH₃ of LA), 2.31 (t, CH₂CO), 4.26-4.38 (m, 2CH₂O of EG), 5.13-5.23 (m, CHO of LA).

Synthesis of stearic acid-ethylene glycol-lactide-succinic anhydride (ST-EG-LA5-SA) (P169): A mixture of (ST-EG-LA5) (40 g, 1 mole equ.), succinic anhydride (3.81 g, 1 mole equ.) and tin(II) 2-ethylhexanoate (0.5 wt. %) was heated at 160° C. for 1 h. The reaction was cooled to r.t. to give the product with a yield of 96%. ST-EG-LA5-SA ¹H-NMR (CDCl₃): δ (ppm)=0.87 (t, CH₃), 1.23-1.28 (b, 14 CH₂), 1.46-1.60 (m, CH₂CH₂CO and CH₃ of LA), 2.30 (t, CH₂CO), 2.64, 2.74 (m, 2 CH₂ of SA), 4.28-4.38 (m, 2CH₂O of EG), 5.12-5.23 (m, CHO of LA).

Synthesis of Stearate-Lactide-EG-Lactide-Stearate (ST-EG-LA-ST) (MH36): In a first step, A mixture of lactide (10 g, 5 mole equ.), ethylene glycol (0.88 g, 1 mole equ.) and tin-2-ethylhexanoate (0.5 wt. %) was heated at 170° C. for 3 h. In a second step, a mixture of stearic acid (7.92 g, 2 mole equ. to product of first step) and zinc acetate (2 wt. %) was added to the product of first step and heated at 170° C. for 10 h. ST-EG-LA-ST ¹H-NMR (CDCl₃): δ (ppm)=0.88 (t, 2 CH₃), 1.23-1.30 (b, 28 CH₂), 1.47-1.69 (m, CH₂CH₂CO and CH₃ of LA), 2.29-240 (m, CH₂CO), 4.26-4.39 (m, 2CH₂O of EG), 5.02-5.21 (m, CHO of LA).

### Example 9: Synthesis of Unsaturated, Carboxylic Synthetic Wax

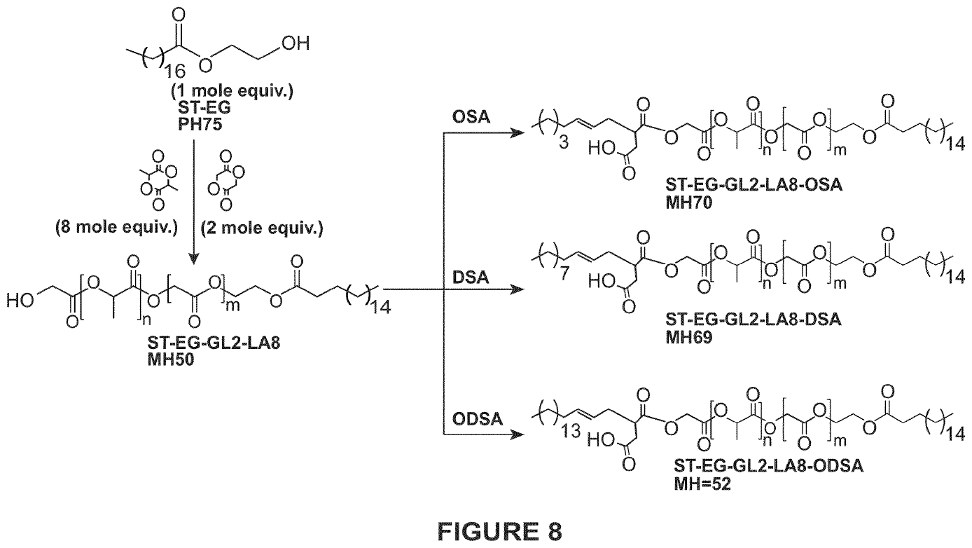

Synthetic waxes were formed by reacting stearate-EG with lactide and/or glycolide, along with 2-octen-1-ylsuccinic anhydride (OSA), 2-(dodecen-1-yl) succinic anhydride (DSA), or (2-octadecen-1-yl) succinic anhydride (ODSA). The OSA, DSA, and ODSA reactants provided unsaturated (C═C) groups and carboxylic (COOH) groups permitting the synthetic wax to be formulated as a high-melting wax (e.g., via polymerizing and/or crosslinking with the unsaturated groups and/or metal complexation with the carboxylic groups). All products were characterized by 1H NMR spectroscopy (500 MHz, Varian 7600-AS, USA). The samples were prepared by dissolving 2-3 mg of each sample in 0.7 mL of chloroform-d (CDCl₃). The general synthetic methods are illustrated in FIG. 8 and described in more detail below.

Synthesis 2-hydroxyethyl stearate (ST-EG) (PH75): A mixture of stearic acid (50 g, 1 mole equiv.), ethylene glycol (218 g, 20 mole equiv.) and zinc acetate dihydrate (2 wt. %) was heated at 200° C. for 15 h. The reaction was cooled to r.t. (about 25° C.), water was added to remove the excess of ethylene glycol, and the product was extracted over chloroform with a yield of 98%. ST-EG ¹H-NMR (CDCl₃): δ (ppm)=0.87 (t, CH₃), 1.24-1.29 (b, 14 CH₂), 1.59-167 (m, CH₂ CH₂CO), 2.34 (t, CH₂CO), 3.83 (t, CH₂OH of EG), 4.19 (t, CH₂OCO of EG).

Synthesis of Stearate-EG-Glycolide2-Lactide8 (ST-EG-GL2-LA8) (MH50): A mixture of ST-EG (1 mole equ.), glycolide (2 mole equ.), L-lactide (8 mole equ.), and tin-2-ethylhexanoate (0.5 wt. %) was heated at 170° C. for 3 h in sealed pressure tube and cooled to r.t. to give the product a yield of 99%.

Reaction of ST-EG-GL2-LA8 with OSA, DSA and/or ODSA to synthesize of ST-EG-GL2-LA8-OSA (MH70), ST-EG-GL2-LA8-DSA (MH69) and ST-EG-GL2-LA8-ODSA (MH52): A mixture of ST-EG-GL2-LA8 (1 mole equ.), OSA, DSA and/or ODSA (1 mole equ.) and tin-2-ethylhexanoate (0.5 wt. %) was heated at 170° C. for 1 h to give the products with yields of about 98%. ST-EG-GL2-LA8-OSA ¹H-NMR (CDCl₃): δ (ppm)=0.88 (t, 2 CH₃), 1.25-1.30 (b, 17 CH₂), 1.57-1.67 (m, CH₂CH₂CO and CH₃ of LA), 1.97-2.02 (m, CH₂CH═CHCH₂), 2.30-3.01 (m, 2 CH₂CO, CHCOOH), 4.28-4.38 (m, 2CH₂O of EG), 4.61-4.90 (m, CH₂O of GL), 5.14-5.23 (2 CHO of LA), 5.29-5.53 (m, CH═CH). ST-EG-GL2-LA8-DSA ¹H-NMR (CDCl₃): δ (ppm)=0.87 (t, 2 CH₃), 1.24-1.31 (b, 21 CH₂), 1.57-1.68 (m, CH₂CH₂CO and CH₃ of LA), 1.95-2.01 (m, CH₂CH═CHCH₂), 2.30-3.02 (m, 2 CH₂CO, CHCOOH), 4.27-4.39 (m, 2CH₂O of EG), 4.60-4.90 (m, CH₂O of GL), 5.13-5.25 (2 CHO of LA), 5.29-5.55 (m, CH═CH). ST-EG-GL2-LA8-ODSA ¹H-NMR (CDCl₃): δ (ppm)=0.87 (t, 2 CH₃), 1.24-1.31 (b, 27 CH₂), 1.48-1.68 (m, CH₂CH₂CO and CH₃ of LA), 1.94-2.04 (m, CH₂CH═CHCH₂), 2.30-3.16 (m, 2 CH₂CO, CHCOOH), 4.28-4.35 (m, 2CH₂O of EG), 4.58-4.90 (m, CH₂O of GL), 5.13-5.25 (2 CHO of LA), 5.43-5.62 (m, CH═CH).

### Example 10: Synthesis of Emulsifiable Unsaturated, Carboxylic Synthetic Wax

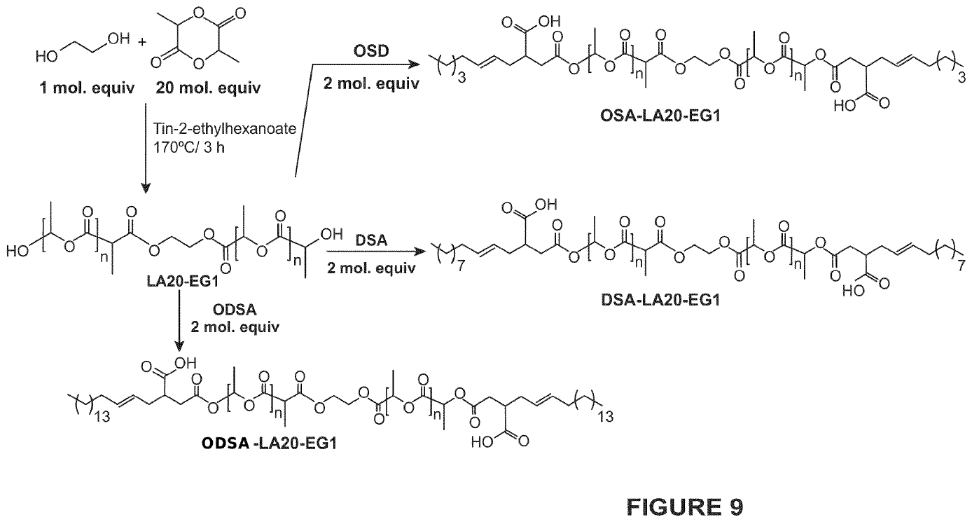

Synthetic waxes were formed by reacting ethylene glycol with lactide, along with 2-octen-1-ylsuccinic anhydride (OSA), 2-(dodecen-1-yl) succinic anhydride (DSA), or (2-octadecen-1-yl) succinic anhydride (ODSA). All products were characterized by 1H NMR spectroscopy (500 MHz, Varian 7600-AS, USA). The samples were prepared by dissolving 2-3 mg of each sample in 0.7 mL of chloroform-d (CDCl₃). The general synthetic methods are illustrated in FIG. 9 and described in more detail below.

Synthesis of LA20-EG1 (MH53): A mixture of L-lactide (20 mole equ., 60 g), ethylene glycol (1 mole equ., 1.30 g) and tin-2-ethylhexanoate (0.5 wt. %) was heated at 170° C. for 3 h in sealed pressure tube and cooled to r.t. (about 25° C.).

Reaction of LA20-EG1 with alkyl succinic anhydrides (MH67, MH55, MH54): A 250 mL round bottle flask was charged with LA20-EG1 (1 mole equ.), OSA, DSA and/or ODSA (2 mole equ.), tin-2-ethylhexanoate (0.5 wt. %) and heated at 170° C. for 1 h to give OSA-LA20-EG1, DSA-LA20-EG1 and ODSA-LA20-EG1, respectively. OSA-LA20-EG1 ¹H-NMR (CDCl₃): δ (ppm)=0.75-0.83 (m, 2 CH₃), 1.13-1.26 (b, 6 CH₂), 1.35-1.60 (m, CH₃ of LA), 1.83-1.94 (m, CH₂CH═CHCH₂), 2.12-2.96 (m, CH₂CO, CHCOOH), 4.21-4.27 (m, 2CH₂O of EG), 4.99-5.07 (CHO of LA), 5.17-5.53 (m, CH═CH). DSA-LA20-EG1 ¹H-NMR (CDCl₃): δ (ppm)=0.86-0.94 (m, 2 CH₃), 1.24-1.34 (b, 14 CH₂), 1.48-1.67 (m, CH₃ of LA), 1.94-2.04 (m, CH₂CH═CHCH₂), 2.26-3.03 (m, CH₂CO, CHCOOH), 4.31-4.37 (m, 2CH₂O of EG), 5.01-5.18 (CHO of LA), 5.26-5.63 (m, CH═CH). ODSA-LA20-EG1 ¹H-NMR (CDCl₃): δ (ppm)=0.85-0.94 (m, 2 CH₃), 1.24-1.32 (b, 26 CH₂), 1.48-1.67 (m, CH₃ of LA), 1.94-2.01 (m, CH₂CH═CHCH₂), 2.28-3.17 (m, CH₂CO, CHCOOH), 4.31-4.38 (m, 2CH₂O of EG), 5.01-5.21 (CHO of LA), 5.30-5.61 (m, CH═CH).

The prepared samples contain free carboxylic groups (—COOH) which can be neutralized easily and emulsified in inorganic salt solution. The three waxy samples OSA-LA20-EG1, DSA-LA20-EG1 and/or ODSA-LA20-EG1 showed 100% emulsification in warmed salty/deionized water solution after 10 min. The samples additionally were tested by adding 100 mg of the waxy samples to water along with NaHCO₃ and/or Na₂CO₃ (10 mg) solution and heated for 5-10 min at NaHCO₃ at 75° C. The samples displayed 100% emulsification in sodium carbonate after 5 min, and the complete emulsification reached 100% with sodium bicarbonate after 10 min.

### Example 11: Synthesis of Recyclable Stearate-Based Synthetic Waxes

This example illustrates the synthesis and performance evaluation of bio-based and biodegradable synthetic waxes with the potential for repulpability and recyclability. The biodegradation and repulping of these waxes are ensured by incorporating lactic/glycolic linkages into their structure. The obtained waxes are applied to kraft paper, and the resulting coated paper is evaluated for its thermal, mechanical, and water/oil resistance. In addition, the synthetic waxes were also blended with a few percent of natural waxes and evaluated for their better performance. Furthermore, the degradability of the waxes was also evaluated under mildly alkaline conditions to assess their suitability for repulping.

Natural waxes such as beeswax and carnauba consist of ester linkages that make them readily biodegradable. Natural waxes have general structures of R¹ (CO)—OR₂ where R₂ represents a long alkyl groups, such as a C30 chain, which provide properties such as water repellency and high melting points. The synthetic wax in this example is similar in behavior to natural waxes, because it incorporates oligomeric lactic acid (PLA) between stearate (C17) chains to compensate for the lack of a C30 chain. In certain cases, glycolic linkages are introduced to further speed up the degradation and hence facilitate repulping, as glycolic linkages can rapidly hydrolyze under mild conditions. By changing the molecular weight of oligo-PLA, the melting temperature of the synthetic waxes as well as water and oil resistance can be adjusted or controlled.

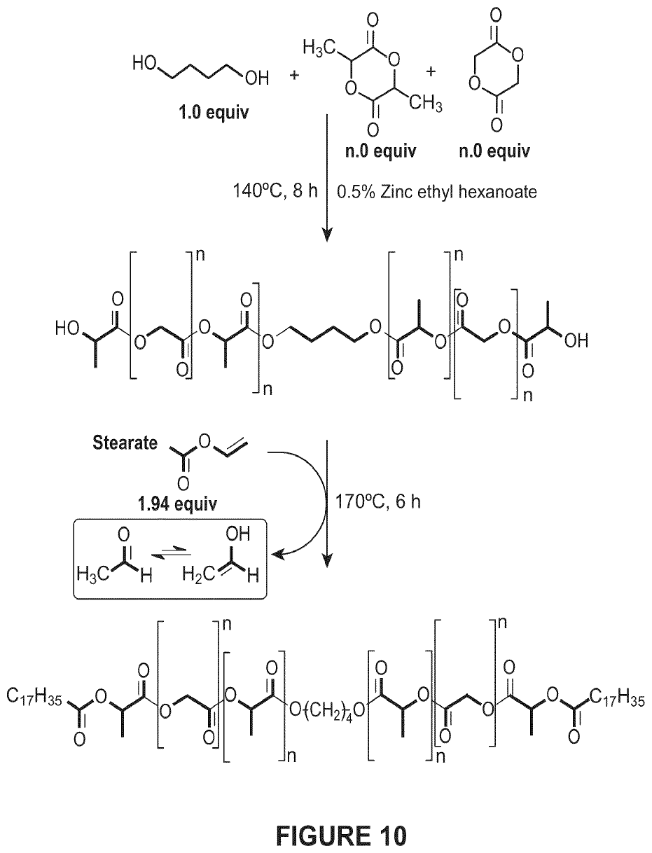

FIG. 10 illustrates PLA-stearate-based biodegradable synthetic waxes with and without glycolic units. A two-step approach is used to synthesize these waxes. First, lactide (and optionally glycolide) were subjected to a ring-opening polymerization, using 1,4-butanediol in the presence of 0.5 wt % catalyst Zn(II) 2-ethylhexanoate. Zn(II) 2-ethylhexanoate was used because of its low toxicity as well as its good miscibility. In the next step, the obtained poly(lactic acid) and poly(lactide-co-glycolide) (PLGA) were further reacted with vinyl stearate to attach stearate moieties to both ends of the oligomer.

Table 9 shows the relative proportions of butane diol, lactide, glycolide, and vinyl stearate in different synthesized samples. The obtained wax with three lactide units was viscous even at room temperature because of the small molecular chain of PLA in the system. Adding four lactide units to the system resulted in a relatively hard wax compared to the previous sample, but relatively poor water resistance performance on starch-coated kraft paper. In subsequent reactions, the number of lactide units gradually increased from five to seven in the system, which offered excellent water and grease resistance in coated materials, thus making it ideal for paper applications. However, a further increase in the number of lactide units resulted in a higher melting wax but weakened water and grease resistance. This could be due to the brittleness of the coating materials, which may lead to some invisible cracks where water or oil can penetrate.

TABLE 9
Proportions of Butanediol, Lactide,
Glycolide, and Stearate in Recyclable Waxes

| | | | Lactide | Glycolide | Vinyl |
|---|---|---|---|---|---|
| Sample | Name | Butanediol | Units | Units | Stearate |
| 1 | W-L3 | 1.0 eq. | 3 | — | 1.94 eq. |
| 2 | W-L4 | 1.0 eq. | 4 | — | 1.94 eq. |
| 3 | W-L5 | 1.0 eq. | 5 | — | 1.94 eq. |
| 4 | W-L5G1 | 1.0 eq. | 5 | 1 | 1.94 eq. |
| 5 | W-L6 | 1.0 eq. | 6 | — | 1.94 eq. |
| 6 | W-L7 | 1.0 eq. | 7 | — | 1.94 eq. |
| 7 | W-L8 | 1.0 eq. | 8 | — | 1.94 eq. |
| 8 | W-L9 | 1.0 eq. | 9 | — | 1.94 eq. |

The polymeric waxes were variously tested for melting properties via differential scanning calorimetry (DSC). The polymeric waxes were also coated on a 5% starch-coated kraft paper substrate and tested for their resistance properties, including cobb1800 water resistance and kit rating oil/grease resistance. The coatings were formed by dissolved 1 g of wax in 2 mL of ethyl acetate or hexane before being applied to the starch-coated kraft paper using an applicator rod. The coated samples were subsequently dried for 3-4 h before they were subjected to further analysis. Solvents were only used to provide uniform thickness and ensure a smooth coating process. The resistance properties were compared with uncoated kraft paper (“KR”), kraft paper with 5% starch coating (“KRS”) (but no wax coatings), 5% starch-coated kraft paper with carnauba wax coating (“KRS-carnauba”), and 5% starch-coated kraft paper with paraffin wax (“KRS-paraffin”) coating as control samples. The results are shown in Table 10.

TABLE 10
Melting and Resistance Values and for Recyclable Synthetic Waxes

| Sample | DSC mp (° C.) | Cobb1800 (g/m²) | Kit |
|---|---|---|---|
| KR | n/a | 140.0 | 0 |
| KRS | n/a | 76.0 | 8 |
| KRS-Carnauba | n/a | 45.6 | — |
| KRS-Paraffin | n/a | 22.0 | — |
| W-L4 | ~37 | 33.2 | 12 |
| W-L5 | ~38 | 8.3 | 12 |
| W-L5G1 | ~45 | 9.0 | 12 |
| W-L6 | ~47 | 7.5 | 12 |
| W-L7 | ~53 | 3.3 | 12 |
| W-L8 | ~41 | 3.4 | 12 |

To demonstrate a solvent-free coating approach for the disclosed waxes, a compression molding technique was used to apply a coating onto paper. In this setup, W-L7 was coated onto paper by applying heat and pressure. Subsequently, the Cobb1800 and kit rating values were determined and were found to be substantially the same and the W-L7 coated paper samples that were prepared via a solution-based method.

The synthesized waxes were blended with carnauba wax (about 90:10 w/w synthesized wax:carnauba wax), which significantly enhanced the water resistance of the coated papers. The Cobb1800 values for most blended wax samples were in the range of 2.0 and 4.0 g/m². These values of blended wax coated samples were significantly lower compared to those that had not been blended with carnauba wax, representing an incredible enhancement in their water resistance, and thus making them compatible with practical packaging applications. For example, the Cobb1800 value recorded for the sample W-L5G1 was primarily 9.0 g/m². However, with the integration of carnauba wax, this value was decreased to 1.9 g/m², which indicates that a total ~90% decrease was attained by the addition of 10% carnauba wax. This decrease in Cobb1800 values indicates that a dramatic improvement in the water resistance was achieved, making the wax-coated paper highly competent in repelling water. The tested W-L4, L5, L6, and L7 blends with carnauba wax had Cobb1800 values of 12.4, 3.4, 4.1, 2.6, and 2.0 g/m², respectively. Furthermore, the addition of carnauba wax to the synthesized waxes also enhanced the oil and grease resistance of the coated samples, maintaining kit rating values of 12/12 for the tested W-L4, L5, L5G1, L6, and L7 blends with carnauba wax.

To evaluate the recyclability of the wax-coated paper samples, chemical degradation of some selected wax samples (W-L7 and W-L5G1) was tested in an aqueous solution of 5 wt % Na₂CO₃ (sodium bicarbonate) at 50° C. for 3 minutes. This temperature was selected because 50-55° C. is widely used as the temperature range for paper repulping processes. This reaction likely involves the cleavage of ester linkages between the PLA chain and stearate groups, thus leading to the disintegration of the wax structure to their components. The W-L7 and W-L5G1 samples turned into a milky solution within 3 min at 50° C. The 1H NMR spectrum of the hydrolyzed products (emulsion) revealed that lactic and glycolic salts were obtained. This behavior of an efficient and rapid hydrolysis process demonstrates the susceptibility of the synthetic wax materials to efficient chemical degradation under mild conditions. In contrast, when the paraffin and carnauba waxes were subjected to similar hydrolysis reactions, no emulsion or degradation was observed. In fact, the paraffin and carnauba waxes remained fully intact.

A wax-coated paper sample (W-L7) was reacted with 2% Na₂CO₃ at 65 to 70° C. for 5 min. The recovered paper was then washed with distilled water to ensure that any residues from the coating material were completely eliminated and subsequently it was dried in oven at 70 to 80° C. The recovered paper pulp was then characterized via FTIR analysis, and this pulp exhibited a similar spectrum to that of the uncoated kraft paper, suggesting the absence of any wax and validating a successful repulping of synthetic wax-coated paper according to the disclosure.

### Example 12: Repulpability and Recyclability of Synthetic Wax-Coated Paper

This example illustrates the repulpability and recyclability of paper coated with synthetic waxes according to the disclosure. Paper samples coated with a synthetic wax were reacted with 2% Na₂CO₃ at 65 to 70° C. for 5 min to remove the wax coating. The synthetic waxes used included ST-EG-LA5-SA) (P169; Example 8 and FIG. 7), DSA-LA20-EG1 (MH55; Example 10 and FIG. 9; alternatively DSA-LA10-EG1-LA10-DSA), and ST-EG-GL2-LA8-OSA (MH70; Example 9 and FIG. 8). The recovered paper was then washed with distilled water to ensure that any residues from the coating material were completely eliminated and subsequently it was dried in oven at 70 to 80° C. The recovered paper pulp was then characterized via FTIR analysis, and this pulp exhibited a similar spectrum to that of the uncoated kraft paper, suggesting the absence of any wax and validating a successful repulping of synthetic wax-coated paper according to the disclosure.

Repulpability Testing Method: Repulpability was evaluated using the FBA Voluntary Standard for Repulping and Recycling Corrugated Fiberboard Treated to Improve Its Performance in the Presence of Water and Water Vapor—Part I Repulpability. Briefly, paper samples are repulped in a Modified Waring Blender and a British Disintegrator in water at a pH of 7 (+/−0.5 pH units) that is maintained at 125° F. (+/−10° F.; about 52° C.+/−6° C.). The pulped material is separated in a screen with 0.010 inch (about 0.0254 cm) slots to determine fiber recovery as a percentage of the amount of fiber charged. 85% repulping yield is required to pass this test. The yield of repulping is calculated as the fraction of fiber accepted (or recovered on screen) relative to total fiber accepted (or recovered on screen) plus fiber rejected (or passing through screen). The results are shown in Table 11.

TABLE 11
Repulpability of Synthetic Wax-Coated Paper

| Content | Unit | Wax P169 | Wax MH55 | Wax MH70 |
|---|---|---|---|---|
| Moisture | wt % | 6.40 | 6.65 | 7.38 |
| Sample charged | g | 22.6 | 22.4 | 22.1 |
| Screen reject | g | 0.60 | 0.22 | 0.07 |
| Screen accepts | g | 17.76 | 19.29 | 18.57 |
| Yield of sample | wt % | 96.73 | 98.87 | 99.62 |
| Pass/Fail | | Pass | Pass | Pass |

Recyclability Testing Method: A lab-scale recyclability test was performed. The test procedure is modified according to FBA Voluntary Standard for Repulping and Recycling Corrugated Fiberboard Treated to Improve Its Performance in the Presence of Water and Water Vapor-Part II Recyclability. Briefly, 20% coated sample and 80% uncoated base paper is mixed and repulped in a lab-scale pulper at pH 7 and 125° F. (about 52° C.). The pulped suspension is passed through a vibration flat screen with 0.010 inch (about 0.0254 cm) slots. Handsheets are made from screen accepts. Properties including Coefficient of Friction (Slide Angle), Short Span Compression Strength (STFI), Burst Strength, Water-Drop Penetration, and Stickies are investigated following TAPPI standards. The results are compared to a control sample, which is a 100% base paper pulped and screened using identical conditions.

The following test reference methods were used: TAPPI T205 (Making Handsheets), TAPPI T815 (Coefficient of Static Friction (Slide Angle)), TAPPI T831 (Water Drop Penetration Test), TAPPI T826 (Short Span Compression Strength (STFI)), TAPPI T403 (Burst Strength), and TAPPI T277 (Stickies Count). The following evaluation criteria were used: (1) Appearance of the handsheets made from the test sample should show no substantial difference from that of control sheets and the stickies count is less than 15 counts, or not exceeding 30% greater counts than the control. (2) The decrease in the slide angle of the handsheets made from test sample should not be greater than 15%. (3) STFI and burst strength of the handsheets made from the test sample, normalized to the basis weight, should show no more than a 10% decrease for the respective values for the control. (4) The water drop penetration of handsheets made from the test sample should not exceed the water drop penetration of the control by more than 200 seconds. The results are shown in Table 12.

TABLE 12
Recyclability of Synthetic Wax-Coated Paper

| | | Base | Wax | Wax | Wax |
|---|---|---|---|---|---|
| Quantity | Unit | Paper | P169 | MH55 | MH70 |
| Basis weight | g/m² | 100 | 101 | 110 | 105 |
| Coefficient | Degrees | 32.4 | 31.4 | 34.0 | 28.4 |
| of Friction | | | | | |
| Water drop | Sec | 2.0 | 2.0 | 1.5 | 1.5 |
| penetration | | | | | |
| Stickies | Counts | 35 | 55 | 72 | 88 |
| Burst strength | kN/m² | 137 | 148 | 157 | 155 |
| Burst strength | Index | 0.198 | 0.213 | 0.207 | 0.214 |
| | value | | | | |
| STFI | N/m | 790 | 869 | 904 | 869 |
| STFI | Index | 0.045 | 0.049 | 0.047 | 0.047 |
| | value | | | | |

All samples met the requirements for strength, slide angle, and water penetration. No decrease in burst strength or STFI strength was observed on any of the samples. No significant increase in water penetration was observed on any of the samples. Decrease in coefficient of friction was observed, however, the decrease was less than 15%. P169 had 57% more stickies than control, MH55 had 106% more stickies, and MH70 had 154% more stickies.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

Throughout the specification, where the compositions, processes, kits, or apparatus are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

1.-36. (canceled)

37. A synthetic wax according to the following Formula IA or IB: 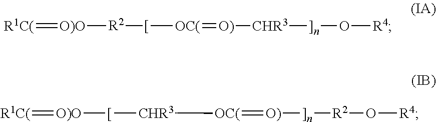
wherein:
R¹ is a hydrocarbon group having 11 to 39 carbon atoms;
R² is a hydrocarbon linking group having 2 to 18 carbon atoms;
n is 4 to 50;
R³ is independently H or CH₃ for each of the n repeat units; and
R⁴ is H or hydrocarbon group having 2 to 40 carbon atoms, with the proviso that when R⁴ is H, then R¹ contains at least one of a carboxylic group and an unsaturated carbon-carbon double bond.

38. The synthetic wax of claim 37, wherein n is 4 to 20.

39. The synthetic wax of claim 38, wherein:
R¹ is C₁₇H₃₅;
R² is C₂H₄; and
OR⁴ is OC(═O)—C₁₇H₃₅.

40. The synthetic wax of claim 38, wherein R¹ is selected from the group consisting of:
(i) R^{1A}, and
(ii) R^{1A}-d-;
wherein:
R^{1A} is a C₈ to C₃₉ alkyl group or unsaturated analog thereof; and
d is a hydrocarbon linking group containing 1 to 4 carbon atoms and a pendant carboxylic acid group or salt thereof.

41. The synthetic wax of claim 38, wherein OR⁴ is selected from the group consisting of:
(i) —OC(═O)—R⁵,
(ii) —OC(═O)-d-R⁵ or a salt thereof,
(iii) —OC(═O)—CH═CH₂,
(iv) —OC(═O)—C(CH₃)═CH₂,
(v) —OC(═O)—C(COOH)═CH₂,
(vi) —[—OC(═O)—CHR₃—]ₘ—OC(═O)—R⁵ in the synthetic wax according to Formula IB,
(vii) —[—OC(═O)—CHR₃—]ₘ—OC(═O)-d-R⁵ or a salt thereof in the synthetic wax according to Formula IB, and
(viii) combinations thereof;
wherein:
R⁵ is a C1 to C39 alkyl group or unsaturated analog thereof;
d is a hydrocarbon linking group containing 1 to 4 carbon atoms and a pendant carboxylic acid group or salt thereof; and
m is 4 to 20.

42. The synthetic wax of claim 38, comprising at least one synthetic wax according to Formula IA.

43. The synthetic wax of claim 38, comprising at least one synthetic wax according to Formula IB.

44. The synthetic wax of claim 38, comprising:
at least one synthetic wax according to Formula IA; and
at least one synthetic wax according to Formula IB.

45. The synthetic wax of claim 38, wherein:
the synthetic wax has a melting temperature in a range of 30° C. to 90° C.; and
the synthetic wax has a molecular weight in a range of 300-5000 g/mol.

46. The synthetic wax of claim 38, wherein:
the synthetic wax has a melting temperature in a range of 30° C. to 160° C.; and/or
the synthetic wax has a molecular weight in a range of 2000-100000 g/mol.

47. A synthetic wax composition comprising:
a synthetic wax according to claim 38; and
one or more additives blended with the synthetic wax;
wherein:
the additives are present in an amount of 1 wt. % to 50 wt. % relative to the synthetic wax composition; and
the additives are selected from the group consisting of inorganic fillers, polymeric fillers, nanoparticles, natural waxes, and combinations thereof.

48. A synthetic wax dispersion comprising:
an aqueous medium; and
a synthetic wax according to claim 38 dispersed in the aqueous medium.

49. A coated article comprising:
a substrate; and
a coating on the substrate, the coating comprising the synthetic wax of claim 38.

50. The coated article of claim 49, wherein:
the substrate is a cellulosic substrate;
the coated article has a kit rating in a range of 4 to 12;
the coated article has a cobb rating of 20 g/m² or less; and
the coating has at least one of properties (I), (II), and (III):
(I) the coated article has a relative permeability for water vapor of 0.5 or less, relative to a corresponding substrate without the coating thereon;
(II) the coated article has a water contact angle in a range of 80° to 120° for a 10 μL deionized water droplet measured 30 sec after application of the droplet; and
(III) the coated article has an oil contact angle in a range of 40° to 75° for a 10 μL castor oil droplet measured 30 sec after application of the droplet.

51. A method for degrading a synthetic wax, the method comprising:
contacting the synthetic wax according to claim 38 with at least one of a carbonate salt, a bicarbonate salt, and an aqueous solution thereof at a temperature and for a time sufficient to degrade the synthetic wax.

52. A high-melting synthetic wax comprising:
a polymerization reaction product of a synthetic wax according to claim 38 in which at least one of R¹ and R⁴ contains at least one carbon-carbon double bond.

53. A high-melting synthetic wax comprising:
a complex between a polyvalent metal cation and a synthetic wax according to claim 38 in which at least one of R¹ and R⁴ contains at least one carboxylic group.

54. A synthetic wax according to the following Formula I: 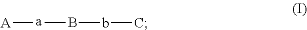
wherein:
A is a hydrocarbon ester group having 12 to 40 carbon atoms;
B is an oligomeric residue of (i) n glycolic acid units, (ii) n lactic acid units, or (iii) n glycolic acid units and lactic acid units in total, where n is 4 to 50;
C is either OH or a hydrocarbon ester group having 2 to 40 carbon atoms, with the proviso that when C is OH, then A contains at least one of a carboxylic group and an unsaturated carbon-carbon double bond;
a is either absent, or present as a linking group between A and B; and
b is either absent, or present as a linking group between B and C.

55. The synthetic wax of claim 54, wherein n is 4 to 20.

56. The synthetic wax of claim 55, wherein at least one of a and b is present.
